SEQUENCE LISTING (24 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 13 aa, Severe acute respiratory syndrome coronavirus 2
MFVFLVLLPLVSS

SEQ ID NO: 3 - protein, 18 aa, Artificial Sequence
  source: /note=“Description of Artificial Sequence Synthetic peptide”
MPLYKLLNVLWLVAVSNA

SEQ ID NO: 4 - protein, 1198 aa, Severe acute respiratory syndrome coronavirus 2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIK

SEQ ID NO: 5 - protein, 4 aa, Severe acute respiratory syndrome coronavirus 2
RRAR

SEQ ID NO: 6 - protein, 4 aa, Artificial Sequence
  source: /note=“Description of Artificial Sequence Synthetic peptide”
GSAS

SEQ ID NO: 7 - protein, 27 aa, Unknown
  source: /note=“Description of Unknown Foldon sequence”
GYIPEAPRDGQAYVRKDGEWVFLSTFL

SEQ ID NO: 8 - DNA, 81 bp, Unknown
  source: /note=“Description of Unknown Foldon sequence”
ggttatattcctgaagctccaagagatgggcaagcttacgttcgtaaagatggcgaatgggtattcctttctaccttttta

SEQ ID NO: 9 - DNA, 81 bp, Artificial Sequence
  source: /note=“Description of Artificial Sequence Synthetic oligonucleotide”
ggttatataccagaggctcctagagatggccaagcatacgtgcgcaaagatggtgaatgggtctttctcagcacattctta

SEQ ID NO: 10 - protein, 1243 aa, Artificial Sequence
  source: /note=“Description of Artificial Sequence Synthetic polypeptide”
MPLYKLLNVLWLVAVSNAQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGYIPEAPRDGQAYVRKDGEWVFLSTFL

SEQ ID NO: 11 - protein, 17 aa, Unknown
  source: /note=“Description of Unknown Chitinase signal sequence”
MLYKLLNVLWLVAVSNA

SEQ ID NO: 12 - DNA, 41 bp, Artificial Sequence
  source: /note=“Description of Artificial Sequence Synthetic oligonucleotide”
ctgttttcgtaacagttttgtaataaaaaaacctataaata

SEQ ID NO: 13 - protein, 1273 aa, Artificial Sequence
  source: /note=“Description of Artificial Sequence Synthetic polypeptide”
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 14 - protein, 1240 aa, Artificial Sequence
  source: /note=“Description of Artificial Sequence Synthetic polypeptide”
MPLYKLLNVLWLVAVSNAQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGYIPEAPRDGQAYVRKDGEWVFLSTFL

SEQ ID NO: 15 - DNA, 60 bp, Artificial Sequence
  source: /note=“Description of Artificial Sequence Synthetic oligonucleotide”
taaataatgcccttgtacaaattgttaaacgttttgtggttggtcgccgttagtaacgcg

SEQ ID NO: 16 - DNA, 60 bp, Artificial Sequence
  source: /note=“Description of Artificial Sequence Synthetic oligonucleotide”
attgttaaacgttttgtggttggtcgccgttagtaacgcgcagtgtgttaatcttacaac

SEQ ID NO: 17 - DNA, 60 bp, Artificial Sequence
  source: /note=“Description of Artificial Sequence Synthetic oligonucleotide”
ctcctactaaattaaatgatctctgctttactaatgtctatgcagattcatttgtaatta

SEQ ID NO: 18 - DNA, 60 bp, Artificial Sequence
  source: /note=“Description of Artificial Sequence Synthetic oligonucleotide”
ctctgctttactaatgtctatgcagattcatttgtaattagaggtgatgaagtcagacaa

SEQ ID NO: 19 - DNA, 60 bp, Artificial Sequence
  source: /note=“Description of Artificial Sequence Synthetic oligonucleotide”
ttcacaaatattaccagatccatcaaaaccaagcaagaggtcatttattgaagatctact

SEQ ID NO: 20 - DNA, 60 bp, Artificial Sequence
  source: /note=“Description of Artificial Sequence Synthetic oligonucleotide”
catcaaaaccaagcaagaggtcatttattgaagatctacttttcaacaaagtgacacttg

SEQ ID NO: 21 - DNA, 57 bp, Artificial Sequence
  source: /note=“Description of Artificial Sequence Synthetic oligonucleotide”
atgagcaggtatataaatgagtaattaattaagtaccgactctgctgaagaggagga

SEQ ID NO: 22 - DNA, 57 bp, Artificial Sequence
  source: /note=“Description of Artificial Sequence Synthetic oligonucleotide”
gtaattaattaagtaccgactctgctgaagaggaggaaattctccttgaagtttccc

SEQ ID NO: 23 - DNA, 56 bp, Artificial Sequence
  source: /note=“Description of Artificial Sequence Synthetic oligonucleotide”
tattcctttctacctttttataattaattaagtaccgactctgctgaagaggagga

SEQ ID NO: 24 - DNA, 56 bp, Artificial Sequence
  source: /note=“Description of Artificial Sequence Synthetic oligonucleotide”
taattaattaagtaccgactctgctgaagaggaggaaattctccttgaagtttccc

(19) United States
(12) Patent Application Publication
(10) Pub. No.: US 2024/0016918 A1
(43) Pub. Date: Jan. 18, 2024

(54) VACCINES AGAINST SARS-COV-2 INFECTIONS

(71) Applicant: SANOFI PASTEUR INC., Swiftwater, PA (US)

(72) Inventors: Natalie ANOSOVA, Lexington, MA (US); Salvador Fernando AUSAR, Markham (CA); Catherine BERRY, Lyon (FR); Florence BOUDET, Lyon (FR); Danilo CASIMIRO, Harleysville, PA (US); Roman M. CHICZ, Belmont, MA (US); Gustavo DAYAN, Bethlehem, PA (US); Guy DE BRUYN, Bushkill Township, PA (US); Carlos DIAZGRANADOS, Wind Gap, PA (US); Tong-Ming FU, Philadelphia, PA (US); Marie GARINOT, Lyon (FR); Lorry GRADY, Simsbury, CT (US); Sanjay GURUNATHAN, Bethlehem, PA (US); Kirill KALNIN, Pelham, NH (US); Nikolai KHRAMTSOV, Branford, CT (US); Valérie LECOUTURIER, Chazay d'Azergues (FR); Nausheen RAHMAN, Noth York (CA); Sophie RUIZ, Lyon (FR); Stephen SAVARINO, Saylorsburg, PA (US); Saranya SRIDHAR, London (GB); Indresh K. SRIVASTAVA, Marlborough, CT (US); James TARTAGLIA, Nazareth, PA (US); Timothy TIBBITTS, East Greenwich, RI (US)

(73) Assignee: SANOFI PASTEUR INC., Swiftwater, PA (US)

(21) Appl. No.: 18/042,626

(22) PCT Filed: Aug. 23, 2021
(86) PCT No.: PCT/US2021/047152

§ 371 Date: Feb. 23, 2023

Related U.S. Application Data
(60) Provisional application No. 63/069,172, filed on Aug. 24, 2020.
(60) Provisional application No. 63/131,278, filed on Dec. 28, 2020.
(60) Provisional application No. 63/184,065, filed on May 4, 2021.
(60) Provisional application No. 63/201,848, filed on May 14, 2021.

(51) Int. Cl. A61K 39/215; A61P 37/04

(52) U.S. Cl. CPC A61K 39/215; A61P 37/04; A61K 2039/53

(57) ABSTRACT

Provided are novel vaccines for prophylactic treatment of SARS-CoV-2 infections and COVID-19 and methods of making the vaccines.

### CROSS-REFERENCE TO RELATED APPLICATIONS

This application claims priority from U.S. Provisional Applications 63/069,172, filed Aug. 24, 2020; 63/131,278, filed Dec. 28, 2020; 63/184,065, filed May 4, 2021; and 63/201,848, filed May 14, 2021. The disclosures of the aforementioned priority applications are incorporated herein by reference in their entirety.

### FEDERALLY SPONSORED RESEARCH OR DEVELOPMENT

This invention was made with government support under HHSO100201600005I awarded by U.S. Department of Health and Human Services and ASPR-BARDA; and an Other Transaction Agreement (OTA) W15QKN-16-9-1002 issued by the U.S. Army Contracting Command, ACC-NJ and awarded as a joint mission between the Department of Health and Human Services and the Department of Defense. The government has certain rights in the invention.

### SEQUENCE LISTING

The instant application contains a Sequence Listing which has been submitted electronically in ASCII format and is hereby incorporated by reference in its entirety. The electronic copy of the Sequence Listing, created on Aug. 23, 2021, is named 025532_W0003_SL.txt and is 59,582 bytes in size.

### BACKGROUND OF THE INVENTION

Coronaviruses are a family of enveloped, positive-sense, single-stranded RNA viruses that infect a wide variety of mammalian and avian species. The viral genome is packed into a capsid that is comprised of the viral nucleocapsid (N) protein and surrounded by a lipid envelope. Embedded in the lipid envelope are the membrane (M) protein, the envelope small membrane (E) protein, hemagglutinin-esterase (HE), and the spike (S) protein. The S protein mediates viral attachment and entry into cells.

Human coronaviruses (hCoVs) cause respiratory illnesses. Low pathogenic hCoVs infect the upper respiratory tract and cause mild colds. Highly pathogenic hCoVs predominantly infect lower airways and can cause severe, and sometimes fatal, pneumonia such as severe acute respiratory syndrome (SARS-CoV) and Middle East respiratory syndrome (MERS-CoV). Severe pneumonia caused by hCoVs is typically associated with rapid virus replication, massive inflammatory cell infiltration, and elevated pro-inflammatory cytokines and chemokines, resulting in acute lung injury and acute respiratory distress syndrome (see, e.g., Channappanavar and Perlman, Semin Immunopathol (2017) 39(5):529-39).

Severe acute respiratory syndrome coronavirus 2 (SARS-CoV-2), also known as the 2019 novel coronavirus (2019-nCoV), is the seventh known coronavirus to infect humans, after HCoV-229E, HCoV-NL63, HCoV-OC43, HCoV-HKU1, MERS-CoV, and the original SARS-CoV (Zhu et al., N Eng Med. (2020) 382 (8):727-33). Like the SARS-related coronavirus strain implicated in the 2003 SARS outbreak, SARS-CoV-2 is a member of the subgenus Sarbecovirus (Beta-CoV lineage B). SARS-CoV-2 is the cause of the ongoing 2019-21 coronavirus disease (COVID-19) (Chan et al., Lancet (2020) 395(10223):514-23; Xu et al., Lancet Respir Med. (2020) doi:10.1016/S2213-2600(20)30076-X); GenBank: MN908947.3; Gorbalenya et al., bioRxiv (2020) doi:10.1101/2020.02.07.937862). Human-to-human transmission occurs primarily via respiratory droplets and aerosols.

The clinical profile of COVID-19 varies. In the majority of cases, infected individuals may be asymptomatic or have mild symptoms. Among those with symptoms, typical presentations include fever, cough, shortness of breath, anosmia, and fatigue. More severe manifestations include acute respiratory distress syndrome, strokes, and cytokine release syndrome, in some cases resulting in death. Severe illness can occur in healthy individuals of any age, but it predominantly occurs in adults with advanced age or underlying medical comorbidities. Older adults are most commonly affected and suffer a high mortality rate. Comorbidities and other conditions associated with severe illness and mortality include chronic kidney disease, chronic obstructive pulmonary disease (COPD), an immunocompromised state, obesity, serious heart conditions (e.g., heart failure, coronary artery disease, or cardiomyopathies), sickle cell disease, diabetes, hypertension, liver disease, and pulmonary fibrosis. The risk from COVID-19 also varies by country and regionally within countries around the world (see, e.g., de Souza, Nat Hum Behav. (2020) 4:856-865; Chen, Cell Death Dis. (2020) 11:438).

SARS-CoV-2 infects cells through binding to the cell surface protein angiotensin-converting enzyme 2 (ACE2) (Hoffmann et al., Cell (2020) 181(2):271-80; Walls et al., Cell (2020) 181(2):281-92). The virus gains entry into host cells through the S protein. The S protein is a class I fusion protein and is heavily coated with polysaccharides that help the virus evade immune surveillance. The protein is produced through processing of precursor S polypeptides. The precursor polypeptide undergoes glycosylation, removal of the signal peptide, and cleavage by proprotein convertase furin between residues 685 and 686 to produce to two subunits S1 and S2. S1 and S2 remain associated as a protomer. The S protein is a trimer of the protomer, existing in a metastable prefusion conformation. Upon binding of the S1 subunit to the host cell receptor, the S1 subunit is released from the protein. The remaining S2 subunit transits into a highly stable postfusion conformation and facilitates membrane fusion between the virus and the host cell and hence viral entry into the cell (see, e.g., Wrapp et al., Science (2020) 10.1126/science.abb2507; Shang et al., PNAS (2020) 117(21):11727-34).

The S protein is a key target for vaccine development. It is expected that the protein in the prefusion conformation presents the most neutralization-sensitive epitopes (see, e.g., Wrapp, supra). Successful immunization strategies require stable antigens, and attempts to stabilize the SARS-CoV-2 S protein in the prefusion conformation have been described (see, e.g., Xiong et al., Nat Struct Mol Biol. (2020) doi.org/10.1038/s41594-020-0478-5).

The public health crisis caused by COVID-19 continues unabated, especially in developing countries. Variants of SARS-CoV-2 continue to emerge. There remains an urgent need to develop efficacious vaccines that can help combat the continued threat of COVID-19.

### SUMMARY OF THE INVENTION

The present disclosure provides an isolated polypeptide comprising, from N terminus to C terminus, (i) a sequence that is at least 94%, for example, at least 95% (e.g., at least 96, 97, 98, or 99%) identical to residues 19-1243 of SEQ ID NO:10, wherein residues GSAS (SEQ ID NO:6) at positions 687-690 of SEQ ID NO:10 and residues PP at positions 991 and 992 of SEQ ID NO:10 are maintained in the sequence; and (ii) a trimerization domain, wherein the trimerization domain may comprise SEQ ID NO:7. In some embodiments, the polypeptide further comprises at its N-terminus a signal peptide derived from an insect or baculoviral protein (e.g., a chitinase); in further embodiments, the signal peptide comprises SEQ ID NO:3. In certain embodiments, the polypeptide comprises or has a sequence identical to (i) residues 19-1243 of SEQ ID NO:10, or (ii) residues 19-1240 of SEQ ID NO:14.

In one aspect, the present disclosure provides a recombinant SARS-CoV-2 S protein, wherein the protein is a trimer of a recombinant polypeptide described herein. In some embodiments, the protein is a trimer of a polypeptide having a sequence identical to (i) residues 19-1243 of SEQ ID NO:10, or (ii) residues 19-1240 of SEQ ID NO:14.

The present disclosure also provides a nucleic acid molecule encoding a recombinant polypeptide herein, optionally wherein the nucleic acid molecule comprises SEQ ID NO:9.

The present disclosure also provides a baculoviral vector for expressing the polypeptide herein. In some embodiments, expression of the polypeptide is under the control of a polyhedrin promoter in the baculoviral expression vector. The present disclosure further provides a method of producing a recombinant SARS-CoV-2 S protein, comprising introducing the baculoviral vector into insect cells, culturing the insect cells under conditions that allow expression and trimerization of the polypeptide, and isolating the recombinant SARS-CoV-2 S protein from the culture, wherein the recombinant SARS-CoV-2 S protein is a trimer of the polypeptide, without the signal sequence. Also provided is a recombinant SARS-CoV-2 S protein produced by the method.

The present disclosure further provides an immunogenic composition comprising one, two, three, or more recombinant SARS-CoV-2 S proteins described herein and a pharmaceutically acceptable carrier. In some embodiments, the pharmaceutically acceptable carrier is a phosphate-buffered saline (e.g., comprising 7.5 mM phosphate and 150 mM NaCl, pH 7.2) and optionally comprises a surfactant (e.g., polysorbate 20 at a concentration of, for example, 0.005% to 1%, such as 0.2%). In some embodiments, the composition comprises about 2 μg to about 50 μg, for example, about 2 μg to about 45 μg or about 5 μg to about 50 μg (e.g., 2.5, 5, 10, 15, or 45 μg) of the recombinant S protein, or of each of the recombinant S proteins if more than one is included (or together “each of the recombinant SARS-CoV-2 S protein(s)” as used herein when referring to both monovalent and multivalent scenarios). Alternatively, the aforementioned protein amounts are the total protein amount in the composition. When a composition is said to have two or more proteins, it is intended that these proteins are different from each other.

In some embodiments, the immunogenic composition further comprises an adjuvant, wherein the adjuvant is an oil-in-water emulsion and for each dose (in, e.g., about 0.2, 0.25, 0.3, 0.4, 0.5, 0.6, or 0.7 mL) of the immunogenic composition, the immunogenic composition comprises or is prepared by mixing (i) about 2 μg to about 50 μg, for example, about 2 μg to about 45 μg or about 5 μg to about 50 μg (e.g., 2.5, 5, 10, 15, or 45 μg) of each of the recombinant SARS-CoV-2 S protein(s), and (ii) one dose of an adjuvant, wherein each dose of the adjuvant is 0.25 mL in volume and comprises or is prepared by mixing 12.5 mg squalene, 1.85 mg sorbitan oleate (monooleate), 2.38 mg polyoxyethylene cetostearyl ether, and 2.31 mg mannitol in a phosphate-buffered saline, such as one comprising 7.5 mM phosphate and 150 mM NaCl, pH 7.2. Alternatively, the aforementioned protein amounts are the total protein amount in the composition. In some embodiments, the composition comprises one (monovalent) or more (multivalent) different recombinant SARS-CoV-2 S proteins. For example, the composition comprises two (bivalent), three (trivalent), or four (quadrivalent) different recombinant SARS-CoV-2 S proteins.

In some embodiments, the immunogenic composition herein comprising one, two, three, or more recombinant SARS-CoV-2 S proteins and for every dose (in, e.g., 0.2 mL, 0.25 mL, 0.3 mL, 0.4 mL, 0.5 mL, or 0.6 mL) of the composition, the composition comprises or is prepared by mixing 2 μg to 50 μg, for example, about 2 μg to about 45 μg or about 5 μg to about 50 μg (e.g., 2.5, 5, 10, 15, or 45 μg) of each of the recombinant SARS-CoV-2 S protein(s), 0.097 mg monobasic sodium phosphate monohydrate, 0.65 mg dibasic sodium phosphate dodecahydrate (or 0.26 mg dibasic sodium phosphate anhydrous), 2.2 mg sodium chloride, 50-600 (e.g., 55 or 550) μg polysorbate (e.g., polysorbate 20), and about 0.25 mL water (qs. ad 0.25 mL water). Alternatively, the aforementioned protein amounts are the total protein amount in the composition.

In some embodiments, for every 0.25 or 0.5 mL of the immunogenic composition, the composition comprises 2.5 μg of each of the recombinant SARS-CoV-2 S protein(s), optionally wherein the composition comprises two different recombinant SARS-CoV-2 S proteins. Alternatively, the aforementioned protein amounts are the total protein amount in the composition.

In some embodiments, for every 0.25 or 0.5 mL of the immunogenic composition, the composition comprises 5 μg of each of the recombinant SARS-CoV-2 S protein(s), optionally wherein the composition comprises two different recombinant SARS-CoV-2 S proteins. Alternatively, the aforementioned protein amounts are the total protein amount in the composition.

In some embodiments, for every 0.25 or 0.5 mL of the immunogenic composition, the composition comprises 10 μg of each of the recombinant SARS-CoV-2 S protein(s), optionally wherein the composition comprises two different recombinant SARS-CoV-2 S proteins. Alternatively, the aforementioned protein amounts are the total protein amount in the composition.

In some embodiments, each dose of the immunogenic composition is 0.25 mL in volume without an adjuvant, or 0.5 mL in volume with an adjuvant.

In some embodiments, the immunogenic composition comprises a recombinant SARS-CoV-2 S protein comprising residues 19-1243 of SEQ ID NO: 10 and/or a recombinant SARS-CoV-2 S protein comprising residues 19-1240 of SEQ ID NO:14.

The present disclosure also provides an article of manufacture, e.g., a container, containing the immunogenic composition herein. In some embodiments, the container contains a single dose of the immunogenic composition, e.g., containing 0.25 mL or 0.5 mL of the immunogenic composition. In some embodiments, the container is a pre-filled, single-use syringe. In other embodiments, the container contains multiple doses of the immunogenic compositions.

The present disclosure also provides a kit for intramuscular vaccination, wherein the kit comprises two containers, wherein a first container contains a pharmaceutical composition comprising the recombinant SARS-CoV-2 S protein, and a second container contains an adjuvant. The second container does not comprise both tocopherol and squalene or the adjuvant AS03. In some embodiments, the first container comprises one or more doses of the recombinant SARS-CoV-2 S protein(s), wherein each dose of the protein(s) is about 2 to 50, 2 to 45, or 5 to 50 (e.g., 2.5, 5, 10, 15, 20, 30, 40, or 45) μg (in total or separately) provided in 0.25 mL of a phosphate-buffered saline optionally comprising (i) 7.5 mM phosphate and 150 mM NaCl, pH 7.2, optionally the PBS comprises 0.005%-1% (e.g., 0.2%) of polysorbate 20, or (ii) 0.0975 mg monobasic sodium phosphate, 0.26 mg dibasic sodium phosphate anhydrous, 2.2 mg sodium chloride, 50-600 (e.g., 55 or 550) μg polysorbate (e.g., polysorbate 20), and about 0.25 mL water (qs. ad 0.25 mL water). In some embodiments, each antigen dose comprises 2.5, 5, 10, 15, 20, 30, 40, or 45 μg of recombinant SARS-CoV-2 S protein(s) (if more than one protein, in total or separately), optionally wherein the antigen dose comprises (i) a recombinant SARS-CoV-2 S protein comprising residues 19-1243 of SEQ ID NO: 10, (ii) a recombinant SARS-CoV-2 S protein comprising residues 19-1240 of SEQ ID NO:14, or (iii) both (i) and (ii).

In some embodiments, the second container comprises one or more doses of the adjuvant, wherein each dose of the adjuvant is 0.25 mL in volume and comprises 12.5 mg squalene, 1.85 mg sorbitan monooleate, 2.38 mg polyoxyethylene cetostearyl ether, and 2.31 mg mannitol in a phosphate-buffered saline (e.g., comprising 7.5 mM phosphate, 150 mM NaCl, pH 7.2, and optionally polysorbate (e.g., polysorbate 20). The present disclosure further provides a method of making a vaccine kit, comprising providing the antigen component and/or the adjuvant component of the immunogenic composition herein and packaging them into sterile containers. In some embodiments, the method comprises providing the recombinant S protein and the adjuvant of the immunogenic composition and packaging the protein and the adjuvant into separate sterile containers.

The present disclosure further provides a method of preventing or ameliorating COVID-19 in a subject (e.g., a human subject) in need thereof, comprising administering to the subject a prophylactically effective amount of the immunogenic composition. In some embodiments, the prophylactically effective amount may be administered in a single dose or in two or more doses. In some embodiments, the prophylactically effective amount is about 2 to 50 μg per dose, optionally 5, 10, 15, or 45 μg per dose, of the recombinant SARS-CoV-2 S protein(s) (if more than one protein, in total or separately), administered intramuscularly in a single dose or in two or more doses. In some embodiments, the method comprises administering to the subject two doses of the immunogenic composition with an interval of about two weeks to about three months, wherein each dose of the immunogenic composition comprises 5 μg or 10 μg of the recombinant SARS-CoV-2 S protein(s) in total. The interval may be, e.g., about three weeks or about 21 days, or about four weeks or about 28 days, or about one month.

In some embodiments, prior to the administering step, the subject may have been infected with SARS-CoV-2 (e.g., developed COVID-19) or has been vaccinated with a first COVID-19 vaccine. In some embodiments, prior to the administering step, the subject may have been vaccinated with a genetic or subunit vaccine, or a killed vaccine. In some embodiments, prior to the administering step, the subject has been vaccinated with a genetic vaccine comprising an mRNA that encodes a recombinant SARS-CoV-2 S antigen. In some embodiments, the administering step may take place 4 weeks, one month, three months, six months, or one year post-infection (e.g., after recovery) or after the subject is vaccinated with the first COVID-19 vaccine.

In some embodiments, in the method herein, the immunogenic composition may comprise 2.5 or 5 μg of each of the recombinant SARS-CoV-2 S protein(s), with or without an adjuvant.

In some embodiments, the present immunogenic composition is used as a booster vaccine in a subject with a previous SARS-CoV-2 infection, or in a subject who has been vaccinated with a first COVID-19 vaccine against the same or different viral strain. The first vaccine may be a killed vaccine, a subunit vaccine, or a genetic vaccine (e.g., an mRNA or viral vector vaccine). In further embodiments, the genetic vaccine comprises an mRNA that encodes a recombinant SARS-CoV-2 S antigen, optionally wherein the recombinant SARS-CoV-2 S antigen comprises SEQ ID NO:1, 4, 10, 13, or 14, or an antigenic fragment thereof. In certain embodiments, the present immunogenic composition is administered to the subject about 4 weeks, about one month, about two months, about three months, about four months, about five months, about six months, about seven months, about eight months, about nine months, about ten months, about eleven months, about one year, or more post-infection or after the subject is vaccinated with the first COVID-19 vaccine. In some embodiments, the time for booster is about four to about ten months (e.g., about eight months) post-infection (e.g., after recovery from COVID-19) or after the primary vaccination.

Also provided herein are use of the recombinant protein or immunogenic composition for the manufacture of a medicament for prophylactic treatment of COVID-19, optionally in a method as disclosed herein, as well as the recombinant protein or the immunogenic composition for use in prophylactic treatment of COVID-19, optionally in a method as disclosed herein.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a diagram showing the design for Construct 1, which contains a baculoviral expression cassette for a recombinant SARS-CoV-2 S protein. The expression cassette includes a polyhedrin promoter and a coding sequence for a polypeptide containing a chitinase signal sequence (“ss”) and a SARS-CoV-2 S protein ectodomain containing mutations at a putative furin cleavage site at the S1/S2 junction and a double proline substitution in the S2 subunit. FIG. 1 discloses SEQ ID NOs:5 and 6, respectively, in order of appearance.

FIG. 2A is a schematic to depict the assembly of the SapI digested pPSC12DB-LIC transfer plasmid with synthesized gBlock Fragments. The SapI linearized transfer plasmid is shown in grey, polyhedrin promoter green arrow, gBlock fragments colored yellow, blue and orange, and each overlapping sequence is depicted as identical colors (top panel). The final transfer plasmid containing the preS dTM gene is shown in the bottom panel.

FIG. 2B shows the 5′ and 3′ end sequences of the gBlock Fragments (SEQ ID NOs:15-24, respectively, in order of appearance).

FIG. 3 is a diagram illustrating the process for generating a baculoviral construct for expressing a recombinant SARS-CoV-2 S protein. MV: Master Virus.

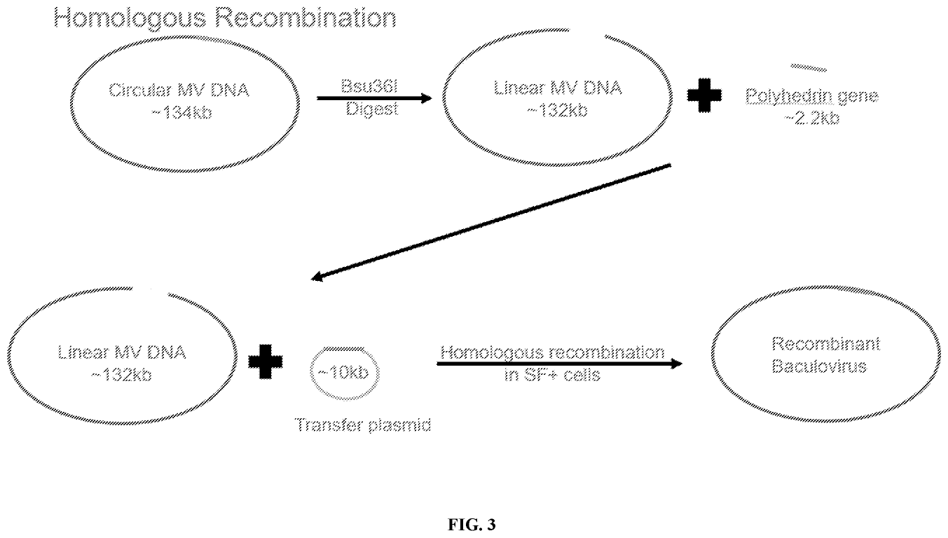

FIG. 4 is a plot showing the D21 and D36 serum S-specific IgG levels in mice injected on D0/D21 with preS dTM and S dTM without adjuvant. Titers are expressed as inverse of dilution for OD=0.2. EU: ELISA units. preS dTM: a recombinant stabilized, prefusion SARS-CoV-2 S protein with deleted transmembrane and cytoplasmic domains (SEQ ID NO:10). S dTM: a recombinant, non-stabilized SARS-CoV-2 S protein with deleted transmembrane and cytoplasmic domains.

FIG. 5 is a plot showing the effect of adjuvant AF03 on S-specific IgG levels in injected mice on Day 21 and Day 36. Titers are expressed as inverse of dilution for OD=0.2. Light colored shapes: Day 21. Dark colored shapes: Day 36.

FIG. 6A is a plot showing neutralizing titers of SARS-CoV-2 infection elicited by the preS dTM vaccine in the absence or presence of AF03 in Swiss Webster mice, on D36. Neutralization is expressed in Plaque Reduction Neutralizing Titers 50% (PRNT₅₀) of serum antibodies obtained from immunized mice on D36. The lower horizontal dash line indicates lower limit of quantitation (LLOQ), which is ½ the starting dilution. The upper horizontal dash line indicates the upper limit of quantitation (ULOQ), which is the highest dilution tested. Y axis is the end point dilution showing 50% reduction in the number of viral plaques counted on a cell monolayer.

FIG. 6B is a plot showing individual S-specific IgG₁ and IgG₂ₐ titers (Log₁₀ EU) elicited by the preS dTM vaccine in the absence or presence of AF03 in Swiss Webster mice, on D36. Bar=mean. Horizontal dotted line=LLOQ.

FIG. 6C is a plot showing individual S-specific IgG₂a/IgG₁ ratio (×100) elicited by the preS dTM vaccine in the presence of AF03 in Swiss Webster mice on D36.

FIG. 6D is a plot showing S1-specific CD4⁺ T cells responses elicited by the preS dTM vaccine in the presence of AF03 in BALB/c mice on D36. Bars: mean %.

FIG. 7 is a plot showing levels of serum IgG against SARS-CoV-2 prefusion S protein in rhesus macaques that were immunized with a targeted dose of 5 or 15 μg of preS dTM with or without the AF03 adjuvant. The IgG levels were measured on D0, D21, and D28. “-” on the X-axis indicates vehicle control. Vehicle is PBS (phosphate-buffered saline). Y axis represents log scale of EU.

FIG. 8 is a plot showing the neutralizing titers of SARS-CoV-2 infection elicited by the preS dTM vaccine in the absence or presence of AF03 in Rhesus macaques on D21 and D28. 50% inhibitory concentration (IC₅₀) titers of neutralizing antibodies were measured against the Integral Molecular SARS-CoV-2 S pseudovirus displaying SARS-CoV-2 S protein from the same study as FIG. 7. The Y-axis represents the Log₁₀ values of the IC₅₀ titers. “Conv”: human SARS-CoV-2 convalescent serum (high titer).

FIG. 9 is a panel of graphs analyzing the S-specific CD4⁺ Th1 profile elicited by the preS dTM vaccine in human PBMCs from 50 human donors, measured in the in vitro MIMIC CD4⁺ lymphoid tissue equivalent (LTE) assay. Secretion of TNF-α, IFN-γ and IL-2 were analyzed. The graphs show the percentage of CD4⁺CD154⁺ cells secreting the three cytokines over the no vaccine condition.

FIG. 10 is a panel of graphs analyzing the S-specific CD4⁺ Th2 profile elicited by the preS dTM vaccine in human PBMCs from 50 human donors, measured in the in vitro MIMIC CD4⁺ LTE assay. Secretion of IL-4, IL-5, and IL-17 were analyzed. The graphs show the percentage of CD4⁺CD154⁺ cells secreting the three cytokines over the no vaccine condition.

FIG. 11 is a pair of graphs showing the decline of neutralizing titers in NHPs on D90 after vaccination with mRNA-VAC2, an mRNA COVID-19 vaccine with a lipid nanoparticle formulation. Groups of cynomolgus macaques (n=4) were vaccinated with mRNA-VAC2 at 15, 45 or 135 μg per dose at D0 and D21, and serum samples collected at the indicated timepoints were tested in pseudovirus (PsV) neutralization assay (panel a) and microneutralization (MN) assay (panel b). Each symbol represents an individual sample and the line geometric means for the group. The neutralization titer of the sample, shown as ID₅₀, was defined as the reciprocal of the highest test serum dilution for which the virus infectivity was reduced by 50% when compared to the assay challenge virus dose. PsV and MN titers of 93 human convalescent (Conv) sera are shown separately in the same scale of Y-axis as other samples.

FIG. 12 is a graph showing robust neutralizing response on D3, 14, 28, 42 after D123 boosting with preS dTM adjuvanted with AF03 (rAg/AF03). Groups of cynomolgus macaques (n=4) were previously vaccinated with mRNA-VAC2 at 15, 45, or 135 μg per dose at D0 and D21. On D123, six NHPs from all prime dose groups were randomized and boosted with 3 μg of rAg/AF03 (n=6). Three control naïve NHPs were immunized with 3 μg of rAg/AF03. Serum samples collected 3 days prior (D-3), 14, 28, and 42 days post-immunizations were tested in MN assay. Each symbol represents an individual sample and the line geometric means for the group. The neutralization titer of the sample, shown as ID₅₀, was defined as in FIG. 11.

FIG. 13 is a graph showing robust binding antibody response after D123 boosting with rAg/AF03. Groups of cynomolgus macaques (n=4) were vaccinated with mRNA-VAC2 at 15, 45, or 135 μg per dose at D0 and D21. On D123, 12 NHPs from all dose groups were randomized and boosted with 3 μg of rAg/AF03 (n=6). Three control naïve NHPs were immunized with 3 μg of rAg/AF03. Serum samples collected 3 days prior (D-3), 14, 28, and 42 days post-immunizations were tested in MN assay. Each symbol represents an individual sample and the line geometric means for the group. The neutralization titer of the sample, shown as ID₅₀, was defined as in FIG. 11.

FIG. 14 is a panel of graphs showing T cell cytokine profiles obtained with PBMCs from NHPs vaccinated with mRNA-VAC1. PBMCs collected at D42 (21 days post the second mRNA-VAC1 injection) were incubated overnight with the SARS-Cov-2 S-protein peptide pools representing the entire S open reading frame. The frequencies of PBMC secreting IFN-γ (left panels) or IL-13 (right panels) were calculated as spots forming cells (SFC) per million PBMC. Each symbol represents an individual sample, and the bar represent the geometric mean for the group. The dotted line represents the lower limit for quantification.

FIG. 15 is a panel of graphs showing T cell cytokine profiles obtained with D171 PBMCs from NHPs vaccinated with mRNA-VAC1 (at D0 and D21) and boosted with rAg/AF03 on D129. PBMCs collected at D42 post boost vaccination were incubated with two peptide pools that represented the entire S open reading frame. The responses of PBMC secreting IFN-γ (top panels) or IL-13 (bottom panels) were calculated as SFC per million PBMC. Each symbol represents an individual sample, and the bars represent the geometric mean for the group. The dotted line represents the lower limit for quantification.

### DETAILED DESCRIPTION OF THE INVENTION

The present disclosure provides immunogenic compositions that are protective against COVID-19. The compositions comprise a recombinant protein derived from the SARS-CoV-2 S protein and expressed in a baculovirus/insect cell expression system. The recombinant protein may comprise an extracellular portion of the S protein (e.g., the entire or part of the S protein ectodomain), while lacking all or part of the transmembrane and cytoplasmic domains of the S protein. The recombinant protein comprises three identical subunit polypeptides (i.e., a homotrimer), each containing a trimerization motif optimized for expression in a baculovirus/insect cell system that facilitates the trimerization of the three subunit polypeptides in a stabilized native prefusion trimer configuration. The immunogenic compositions may comprise the squalene-based AF03 adjuvant (“AF03” hereinafter).

The immunogenic compositions herein can be used for prevention of symptomatic COVID-19 in SARS-CoV-2 naïve human subjects, prevention of moderate-to-severe COVID-19 (e.g., prevention of hospitalization or death), prevention of asymptomatic infection, elicit immunogenicity against homologous matched strain, reduction in viral burden, and/or protection against circulating variant strains. Unless otherwise indicated, a SARS-CoV-2 “variant” refers to a SARS-CoV-2 strain that has amino acid differences in the S protein from the original Wuhan strain (or “D614 strain”; SEQ ID NO:1).

As used herein, the terms “immunogenic composition,” vaccine,” and “vaccine composition” are interchangeable and refer to a composition containing components that can elicit prophylactic protection against SARS-CoV-2 infections, including alleviating COVID-19 symptoms and improving recovery and survival from the disease.

As used herein, percent identity between two amino acid sequences refers to the percentage of amino acid residues in the query sequence that are identical to the residues in the reference sequence, when the query and reference sequences are aligned for maximal identity. The homologous sequence may have the same length as the reference sequence or shorter (e.g., having at least 90% (e.g., at least 91, 92, 93, 94, 95, 96, 97, 98, or 99%) of the length of the reference sequence).

### I. Antigen Components of the Immunogenic Compositions

The immunogenic compositions of the present disclosure comprise a recombinant SARS-CoV-2 S protein. The recombinant protein is stabilized to maintain the native, prefusion trimeric conformation on the viral envelope.

The SARS-CoV-2 S protein has 1273 amino acid residues. An amino acid sequence of the S protein is available under NCBI Accession No. YP_009724390. The sequence is shown below. The signal sequence is boxed (MFVFLVLLPLVSS (SEQ ID NO:2)), and the transmembrane and intracellular domains are underlined. The S1 and S2 junction is between residues 685 and 686, which are in boldface and underlined.

| (SEQ ID NO: 1) | |
|---|---|
| 1 | 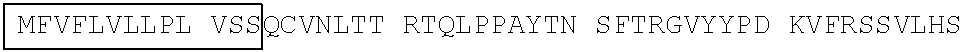 |
| 51 | TQDLFLPFFS NVTWFHAIHV SGTNGTKRFD NPVLPFNDGV YFASTEKSNI |
| 101 | IRGWIFGTTL DSKTQSLLIV NNATNVVIKV CEFQFCNDPF LGVYYHKNNK |
| 151 | SWMESEFRVY SSANNCTFEY VSQPFLMDLE GKQGNFKNLR EFVFKNIDGY |
| 201 | FKIYSKHTPI NLVRDLPQGF SALEPLVDLP IGINITRFQT LLALHRSYLT |
| 251 | PGDSSSGWTA GAAAYYVGYL QPRTFLLKYN ENGTITDAVD CALDPLSETK |
| 301 | CTLKSFTVEK GIYQTSNERV QPTESIVRFP NITNLCPFGE VFNATRFASV |
| 351 | YAWNRKRISN CVADYSVLYN SASFSTFKCY GVSPTKLNDL CFTNVYADSF |
| 401 | VIRGDEVRQI APGQTGKIAD YNYKLPDDFT GCVIAWNSNN LDSKVGGNYN |
| 451 | YLYRLFRKSN LKPFERDIST EIYQAGSTPC NGVEGFNCYF PLQSYGFQPT |
| 501 | NGVGYQPYRV VVLSFELLHA PATVCGPKKS TNLVKNKCVN FNFNGLTGTG |
| 551 | VLTESNKKEL PFQQFGRDIA DTTDAVRDPQ TLEILDITPC SEGGVSVITP |
| 601 | GTNTSNQVAV LYQDVNCTEV PVAIHADQLT PTWRVYSTGS NVFQTRAGCL |
| 651 | IGAEHVNNSY ECDIPIGAGI CASYQTQTNS PRRARSVASQ SIIAYTMSLG |
| 701 | AENSVAYSNN SIAIPTNFTI SVTTEILPVS MTKTSVDCTM YICGDSTECS |
| 751 | NLLLQYGSFC TOLNRALTGI AVEQDKNTQE VFAQVKQIYK TPPIKDFGGF |
| 801 | NFSQILPDPS KPSKRSFIED LLFNKVTLAD AGFIKQYGDC LGDIAARDLI |
| 851 | CAQKFNGLTV LPPLLTDEMI AQYTSALLAG TITSGWTFGA GAALQIPFAM |
| 901 | QMAYRENGIG VTQNVLYENQ KLIANQFNSA IGKIQDSLSS TASALGKLQD |
| 951 | VVNQNAQALN TLVKQLSSNF GAISSVINDI LSRLDKVEAE VQIDRLITGR |
| 1001 | LQSLQTYVTQ QLIRAAEIRA SANLAATKMS ECVLGQSKRV DFCGKGYHLM |
| 1051 | SFPQSAPHGV VFLHVTYVPA QEKNFTTAPA ICHDGKAHFP REGVFVSNGT |
| 1101 | HWFVTQRNFY EPQIITTDNT FVSGNCDVVI GIVNNTVYDP LQPELDSFKE |
| 1151 | ELDKYFKNHT SPDVDLGDIS GINASVVNIQ KEIDRLNEVA KNLNESLIDL |
| 1201 | QELGKYEQYI KWPWYIWLGF IAGLIAIVMV TIMLCCMTSC CSCLKGCCSC |
| 1251 | GSCCKFDEDD SEPVLKGVKL HYT |

The recombinant S protein herein is comprised of three identical polypeptides (“recombinant S polypeptide” herein). Prior to maturation, each recombinant S polypeptide may comprise a signal sequence suitable for protein expression in insect cells. For example, the signal sequence is derived from an insect or baculoviral protein. The signal sequence may also be an artificial signal sequence. In some embodiments, the signal sequence is derived from an insect or baculovirus protein, such as chitinase and GP64. An exemplary chitinase signal sequence is a wildtype chitinase signal sequence

(SEQ ID NO: 11)
MLYKLINVLW LVAVSNA

or a mutant chitinase signal sequence

(SEQ ID NO: 3)
MPLYKLLNVL WLVAVSNA.

A sequence homologous to this chitinase signal sequence (e.g., at least 95, 96, 97, 98, or 99% identical) may also be used, so long as the signal peptide function is retained. See also U.S. Pat. No. 8,541,003.

The recombinant S protein herein comprises a SARS-CoV-2 S protein ectodomain sequence, e.g., the sequence that corresponds to residues 14 to 1,211 of SEQ ID NO:1. An exemplary SARS-CoV-2 S protein ectodomain sequence is shown as follows:

(SEQ ID NO: 4)
QCVNLTTRTQ LPPAYTNSFT RGVYYPDKVF
RSSVLHSTQD LFLPFFSNVT
WFHAIHVSGT NGTKRFDNPV LPFNDGVYFA
STEKSNIIRG WIFGTTLDSK
TQSLLIVNNA TNVVIKVCEF QFCNDPFLGV
YYHKNNKSWM ESEFRVYSSA
NNCTFEYVSQ PFLMDLEGKQ GNFKNLREFV
FKNIDGYFKI YSKHTPINLV
RDLPQGFSAL EPLVDLPIGI NITRFQTLLA
LHRSYLTPGD SSSGWTAGAA
AYYVGYLQPR TFLLKYNENG TITDAVDCAL
DPLSETKCTL KSFTVEKGIY
QTSNFRVQPT ESIVRFPNIT NLCPFGEVEN
ATRFASVYAW NRKRISNCVA
DYSVLYNSAS FSTFKCYGVS PTKLNDLCFT
NVYADSFVIR GDEVRQIAPG
QTGKIADYNY KLPDDFTGCV IAWNSNNLDS
KVGGNYNYLY RLFRKSNLKP
FERDISTEIY QAGSTPCNGV EGENCYFPLQ
SYGFQPTNGV GYQPYRVVVL
SFELLHAPAT VCGPKKSTNL VKNKCVNFNF
NGLTGTGVLT ESNKKFLPFQ
QFGRDIADTT DAVRDPQTLE ILDITPCSFG
GVSVITPGTN TSNQVAVLYQ
DVNCTEVPVA IHADQLTPTW RVYSTGSNVF
QTRAGCLIGA EHVNNSYECD
IPIGAGICAS YQTQTNSPRR ARSVASQSII
AYTMSLGAEN SVAYSNNSIA
IPTNFTISVT TEILPVSMTK TSVDCTMYIC
GDSTECSNLL LQYGSFCTQL
NRALTGIAVE QDKNTQEVFA QVKQIYKTPP
IKDFGGFNFS QILPDPSKPS
KRSFIEDLLF NKVTLADAGF IKQYGDCLGD
IAARDLICAQ KFNGLTVLPP
LLTDEMIAQY TSALLAGTIT SGWTFGAGAA
LQIPFAMQMA YRENGIGVTQ
NVLYENQKLI ANQFNSAIGK IQDSLSSTAS
ALGKLQDVVN QNAQALNTLV
KQLSSNFGAI SSVLNDILSR LDKVEAEVQI
DRLITGRLQS LQTYVTQQLI
RAAEIRASAN LAATKMSECV LGQSKRVDFC
GKGYHLMSFP QSAPHGVVFL
HVTYVPAQEK NFTTAPAICH DGKAHFPREG
VFVSNGTHWF VTQRNFYEPQ
IITTDNTFVS GNCDVVIGIV NNTVYDPLQP
ELDSFKEELD KYFKNHTSPD
VDLGDISGIN ASVVNIQKEI DRLNEVAKNL
NESLIDLQEL GKYEQYIK

In some embodiments, the recombinant S protein may comprise the sequence of SEQ ID NO:4 but for certain amino acid substitutions as further described herein, and is at least 99% (e.g., at least 99.5, 99.6, 99.7, 99.8, 99.9%) identical to SEQ ID NO:4. In further embodiments, the residues at positions 669-672 of SEQ ID NO:4 (in boldface) are changed to residues GSAS (SEQ ID NO:6) and/or the residues at positions 973 and 74 of SEQ ID NO:4 (underlined) are changed to residues PP.

In some embodiments, the recombinant S protein comprises one or more common mutations found in variants circulating in the COVID-19 pandemic. One such mutation is the D614G mutation (numbering according to SEQ ID NO:1) associated with a majority of current COVID-19 incidences around the world. Other mutations that may be included in the recombinant S protein may be one or more of W152C, K417T/N, N440K, V445I, G446A/S, L452R, Y453F, L455F, F456 L, A475V, G476S, T478I/K/A, V483A/F/I, E484Q/K/D/A, F490S/L, Q493 L/R, S494P/L, Y495N, G496 L, P499H, N501Y, V503F/I, Y505W/H, Q506H/K, and P681H mutations (numbering according to SEQ ID NO:1). In some embodiments, the recombinant S protein may include one or more of the mutations N440K, T479I/K/A, and D614G.

In some embodiments, the recombinant S proteins comprises one or more mutations found in SARS-CoV-2 variants, such as B.1.1.7 (British or Alpha variant; e.g., N501Y/P681H/deletion of H69/V70), B.1.351 (South African or Beta variant; e.g., K417N/E484K/N501Y), B1.617 (Indian or Delta variant; e.g., the L452R/E484Q mutations), P.1 (Brazilian or Gamma variant; e.g., K417T/E484K/N501Y), and CAL.20C strain (aka. B.1.429; California or Epsilon variant; e.g., W152C/L452R).

The ectodomain sequence in the recombinant S protein may be modified to improve expression of the protein in host cells (e.g., insect cells) and stability of the produced protein. In some embodiments, the S ectodomain sequence contains a mutation that removes the proprotein convertase (PPC) motif (furin cleavage site) at the junction of the S1 subunit and the S2 subunit. For example, the sequence at the furin cleavage site, RRAR (SEQ ID NO:5; corresponding to residues 682-685 of SEQ ID NO:1) is changed to GSAS (SEQ ID NO:6). Such mutations help preserve the prefusion conformation of the native S protein.

In some embodiments, the ectodomain sequence contains other mutations that help maintain the recombinant S protein in a more stable conformation so as to facilitate antigenic presentation of the prefusion epitopes that are more likely to lead to neutralizing responses. For example, amino acids corresponding to residues 986 and 987 of SEQ ID NO:1 (KV) are mutated to PP (see, e.g., Wrapp, supra; Kirchdoerfer et al., Sci Rep. (2018) 8:15701; Xiong, supra).

The recombinant S protein herein comprises a trimerization domain at the C-terminal region optimized for expression in a baculovirus/insect cell expression system, such that the S protein can assume a stabilized prefusion conformation of the native S protein. The foldon domain coding sequence may be inserted between the last codon and the stop codon of the S ectodomain coding sequence. In some embodiments, the trimerization domain is derived from the foldon domain of T4 phage fibritin (see, e.g., Meier et al., J Mol Biol. (2004) 344(4):1051-69; WO 2018/081318). An exemplary foldon sequence is shown below:

(SEQ ID NO: 7)
GYIPEAPRDG QAYVRKDGEW VFLSTFL.

In some embodiments, the foldon sequence may be optimized to enhance expression of the recombinant protein in host cells. For example, to enhance expression of the recombinant protein in insect cells (e.g., Spodoptera cells), the sequence encoding the foldon sequence may be codon-optimized. The following shows the native coding sequence (top) and a codon-optimized version (bottom) for a foldon domain (nucleotide point mutations are marked by asterisks):

```
                    * * * * * * * *
ggt tat att cct gaa gct cca aga gat ggg caa gct tac gtt cgt
ggt tat ata cca gag gct cct aga gat ggc caa gca tac gtg cgc
G Y I P E A P R D G Q A Y V R
                    * * * * *** * *
aaa gat ggc gaa tcg gta ttc ctt tct acc ttt tta (SEQ ID NO: 8)
aaa gat ggt gaa tcg gtc ttt ctc agc aca ttc tta (SEQ ID NO: 9)
K D G E W V F L S T F L (SEQ ID NO: 7)
```

The recombinant S protein may comprise a tag (e.g., a His tag, a FLAG tag, an HA tag, a Myc tag, or V5 tag) to facilitate purification.

In some embodiments, the recombinant S protein may be a trimer of a polypeptide having the following sequence, but without the signal sequence once processed and assembled. In the sequence below, the signal sequence (residues 1-18) is underlined, the foldon sequence (residues 1217-1243) is double underlined, while mutations relative to the wildtype sequence (artificially introduced) are in boldface and underlined (residues 687-690 and 991-992). This protein is also termed “preS dTM” or “D614 preS dTM” herein.

| (SEQ ID NO: 10) | |
|---|---|
| 1 | MPLYKLLNVL WLVAVSNAQC VNLTTRTQLP PAYTNSFTRG VYYPDKVFRS |
| 51 | SVLHSTQDLF LPFFSNVTWF HAIHVSGTNG TKRFDNPVLP FNDGVYFAST |
| 101 | EKSNIIRGWI FGTTLDSKTQ SLLIVNNATN VVIKVCEFQF CNDPFLGVYY |
| 151 | HKNNKSWMES EFRVYSSANN CTFEYVSQPF LMDLEGKQGN FKNLREFVFK |
| 201 | NIDGYFKIYS KHTPINLVRD LPQGFSALEP LVDLPIGINI TRFQTLLALH |
| 251 | RSYLTPGDSS SGWTAGAAAY YVGYLQPRTF LLKYNENGTI TDAVDCALDP |
| 301 | LSETKCTLKS FTVEKGIYQT SNFRVQPTES IVRFPNITNL CPFGEVFNAT |
| 351 | RFASVYAWNR KRISNCVADY SVLYNSASFS TFKCYGVSPT KLNDLCFTNV |
| 401 | YADSFVIRGD EVRQIAPGQT GKIADYNYKL PDDFTGCVIA WNSNNLDSKV |
| 451 | GGNYNYLYRL FRKSNLKPFE RDISTEIYQA GSTPCNGVEG FNCYFPLQSY |
| 501 | GFQPTNGVGY QPYRVVVLSF ELLHAPATVC GPKKSTNLVK NKCVNFNFNG |
| 551 | LTGTGVLTES NKKFLPFQQF GRDIADTTDA VRDPQTLEIL DITPCSFGGV |
| 601 | SVITPGTNTS NQVAVLYQDV NCTEVPVAIH ADQLTPTWRV YSTGSNVFQT |
| 651 | RAGCLIGAEH VNNSYECDIP IGAGICASYQ TQTNSPGSAS SVASQSIIAY |
| 701 | TMSLGAENSV AYSNNSIAIP TNFTISVTTE ILPVSMTKTS VDCTMYICGD |
| 751 | STECSNLLLQ YGSFCTQLNR ALTGIAVEQD KNTQEVFAQV KQIYKTPPIK |
| 801 | DFGGFNFSQI LPDPSKPSKR SFIEDLLENK VTLADAGFIK QYGDCLGDIA |
| 851 | ARDLICAQKF NGLTVLPPLL TDEMIAQYTS ALLAGTITSG WTFGAGAALQ |
| 901 | IPFAMQMAYR FNGIGVTQNV LYENQKLIAN QFNSAIGKIQ DSLSSTASAL |
| 951 | GKLQDVVNQN AQALNTLVKQ LSSNFGAISS VLNDILSRLD PPEAEVQIDR |
| 1001 | LITGRLQSLQ TYVTQQLIRA AEIRASANLA ATKMSECVLG QSKRVDFCGK |
| 1051 | GYHLMSFPQS APHGVVFLHV TYVPAQEKNF TTAPAICHDG KAHFPREGVE |
| 1101 | VSNGTHWFVT QRNFYEPQII TTDNTFVSGN CDVVIGIVNN TVYDPLQPEL |
| 1151 | DSFKEELDKY FKNHTSPDVD LGDISGINAS VVNIQKEIDR LNEVAKNLNE |
| 1201 | SLIDLQELGK YEQYIKGYIP EAPRDGQAYV RKDGEWVFLS TFL |

A sequence homologous to SEQ ID NO:10 may also be used. For example, a recombinant S polypeptide whose sequence is at least 95% (e.g., at least 96, 97, 98, or 99%) identical to SEQ ID NO:10 may be used. The homologous sequence may have the same length as SEQ ID NO:10 or no more than 10% (e.g., no more than 9, 8, 7, 6, 5, 4, 3, 2, or 1%) shorter or longer than SEQ ID NO:10. In further embodiments, residues GSAS (SEQ ID NO:6) at positions 687-690 of SEQ ID NO:10 and/or residues PP at positions 991 and 992 of SEQ ID NO: 10 are maintained in such a homologous sequence. The percent identity of two amino acid sequences may be obtained by, e.g., BLAST® using default parameters (available at the U.S. National Library of Medicine's National Center for Biotechnology Information website).

In some embodiments, a variant of preS dTM (also “preS dTM variant” herein), i.e., a recombinant S protein containing one or more amino acid differences from SEQ ID NO:10 (e.g., outside the signal sequence region), is used. In further embodiments, the recombinant S protein is derived from the Southern African or Beta variant B.1.351. This variant contains the following mutations (relative to the Wuhan strain or SEQ ID NO: 1): (i) in the NTD domain: L18F, D80A, D215G, L242de1, A243de1, and L244de1; (ii) in the RBD domain: K417N, E484K, N501Y; (iii) in the S1 domain: D614G; and (iv) A701V. The S protein may comprise the following sequence (SEQ ID NO:14), without the signal sequence (underlined; residues 1-18) once processed and secreted from producing cells. The T4 foldon sequence (residues 1214-1240) is double underlined; variations from SEQ ID NO:10 are boxed and boldfaced; and artificially introduced mutations (residues 684-687 and residues 988-989) are underlined and boldfaced). Compared to the S protein derived from the Wuhan strain, this protein also has a deletion of three residues “LAL” immediately after “FQTL” at positions 243-246 below.

| (SEQ ID NO: 14) | |
|---|---|
| 1 | 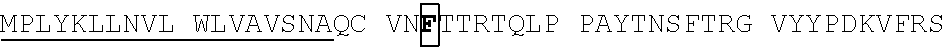 |
| 51 | 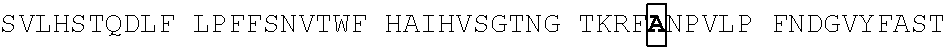 |
| 101 | EKSNIIRGWI FGTTLDSKTQ SLLIVNNATN VVIKVCEFQF CNDPFLGVYY |
| 151 | HKNNKSWMES EFRVYSSANN CTFEYVSQPF LMDLEGKQGN FKNLREFVFK |
| 201 | 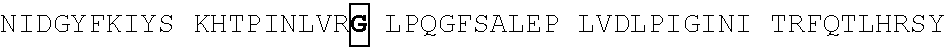 |
| 251 | LTPGDSSSGW TAGAAAYYVG YLQPRTFLLK YNENGTITDA VDCALDPLSE |
| 301 | TKCTLKSFTV EKGIYQTSNF RVQPTESIVR FPNITNLCPF GEVFNATRFA |
| 351 | SVYAWNRKRI SNCVADYSVL YNSASFSTFK CYGVSPTKLN DLCFTNVYAD |
| 401 | 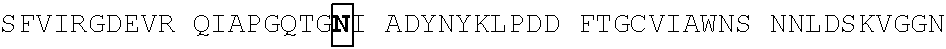 |
| 451 | 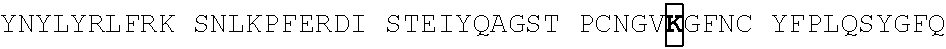 |
| 501 | 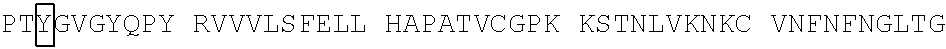 |
| 551 | TGVLTESNKK FLPFQQFGRD IADTTDAVRD PQTLEILDIT PCSFGGVSVI |
| 601 | 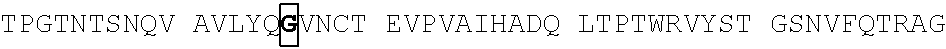 |
| 651 | CLIGAEHVNN SYECDIPIGA GICASYQTQT NSPGSASSVA SQSIIAYTMS |
| 701 | 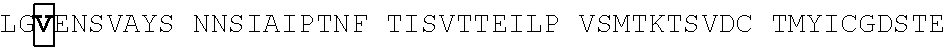 |
| 751 | CSNLLLQYGS FCTQLNRALT GIAVEQDKNT QEVFAQVKQI YKTPPIKDFG |
| 801 | GFNFSQILPD PSKPSKRSFI EDLLFNKVTL ADAGFIKQYG DCLGDIAARD |
| 851 | LICAQKFNGL TVLPPLLTDE MIAQYTSALL AGTITSGWTF GAGAALQIPF |
| 901 | AMQMAYRFNG IGVTONVLYE NOKLIANQFN SAIGKIQDSL SSTASALGKL |
| 951 | QDVVNQNAQA LNTLVKQLSS NFGAISSVLN DILSRLDPPE AEVQIDRLIT |
| 1001 | GRLQSLQTYV TQQLIRAAEI RASANLAATK MSECVLGQSK RVDFCGKGYH |
| 1051 | LMSFPQSAPH GVVFLHVTYV PAQEKNFTTA PAICHDGKAH FPREGVFVSN |
| 1101 | GTHWFVTQRN FYEPQIITTD NTFVSGNCDV VIGIVNNTVY DPLQPELDSF |
| 1151 | KEELDKYFKN HTSPDVDLGD ISGINASVVN IQKEIDRLNE VAKNLNESLI |
| 1201 | DLQELGKYEQ YIKGYIPEAP RDGQAYVRKD GEWVFLSTFL |

In some embodiments, the present immunogenic composition is multivalent (e.g., bivalent, trivalent, or quadrivalent). That is, the composition comprises multiple (e.g., two, three, or four) different recombinant S proteins. One or more of the recombinant S proteins in a multivalent composition may comprise one or more mutations found in SARS-CoV-2 variants, such as D614G and mutations found in newly emergent variant strains, e.g., B.1.1.7, B.1.351, B.1.617, P.1, and CAL.20C.

In some embodiments, the present immunogenic composition is bivalent. In further embodiments, the bivalent composition comprises a first recombinant S protein that is derived from the Wuhan strain and a second recombinant S protein that is derived from the South African strain. In certain embodiments, the bivalent composition comprises a recombinant S protein comprising SEQ ID NO:10, without the signal sequence, and a recombinant S protein comprising SEQ ID NO: 14, without the signal sequence.

### II. Adjuvant Components of the Immunogenic Compositions

The present immunogenic compositions may comprise adjuvants with pharmaceutically acceptable ingredients. The present immunogenic compositions do not comprise both tocopherol and squalene. The present immunogenic compositions also do not comprise the adjuvant AS03 (an oil-in-water emulsion comprising tocopherol and squalene; see, e.g., WO2006/100109; Garçon et al., Expert Rev Vaccines (2012) 11:349-66; Cohet et al., Vaccine (2019) 37(23):3006-21). Adjuvants enhance the magnitude and quality of the immune response to the recombinant S protein. In some embodiments, the present immunogenic compositions may employ an oil-in-water (O/W) emulsion adjuvant that contains squalene but no tocopherol. The adjuvant may promote a balanced Th1/Th2 T helper response. See, e.g., U.S. Pat. Nos. 8,703,095, 9,327,021, and 9,504,659.

Squalene is an oil having the empirical chemical formula C₃₀H₅₀ with six double bonds. This oil is metabolizable and has the required qualities to be used in an injectable pharmaceutical product. It comes from shark liver (animal origin) but can also be extracted from olive oil (plant origin). The amounts of squalene used for the preparation of a concentrated emulsion may be between 0.5% and 5% (e.g., 2.5%).

The O/W squalene-based adjuvant comprises a nonionic hydrophilic surfactant, with a hydrophilic/lipophilic balance (HLB) value no less than 10. Examples of such surfactants are polyoxyethylene alkyl ethers (PAE or POEs), also called polyoxyethylenated fatty alcohol ethers, or n-alcohol polyoxyethylene glycol ethers, or macrogol ethers. These nonionic surfactants are obtained by chemical condensation of a fatty alcohol and ethylene oxide. They have a general chemical formula CH₃(CH₂)ₓ—(O—CH₂—CH₂)ₙ—OH, in which “n” denotes the number of ethylene oxide units (typically 10-60), and (x+1) is the number of carbon atoms in the alkyl chain, typically 12 (lauryl(dodecyl)), 14 (myristyl(tetradecyl)), 16 (cetyl(hexadecyl)), or 18 (stearyl(octadecyl)), so “x” is in the range of from 11 to 17. POEs tend to be mixtures of polymers of slightly varying molecular weights. Accordingly, the emulsions may comprise a mixture of POEs and as such, references made herein to a suitable POE for use in an emulsion, the recited ether is the primary but not necessarily the only POE present in the emulsion. POEs suitable for use can be, at ambient temperature, in liquid or solid form. Suitable solid compounds are those that dissolve directly in the aqueous phase or do not require substantial heating. Insofar as the number of ethylene oxide units is sufficient, lauryl alcohol, myristyl alcohol, cetyl alcohol, oleyl alcohol and/or stearyl alcohol may be used herein. Examples of POEs are ceteareth-12 (e.g., Eumulgin® B1), ceteareth-20 (e.g., Eumulgin® B2), steareth-21 (e.g., Eumulgin® S21), ceteth-20 (e.g., Simulsol™ 58 or Brij®58), ceteth-10 (e.g., Brij® 56), steareth-10 (e.g., Brij® 76), steareth-20 (e.g., Brij® 78), oleth-10 (e.g., Brij® 96 or 97), and oleth-20 (Brij® 98 or 99), where the number attributed to each chemical name corresponds to the number of ethylene oxide units in the chemical formula.

The O/W squalene-based emulsion adjuvant also comprises a nonionic hydrophobic surfactant. Surfactants that are suitable in this regard, include, for example, sorbitan esters or mannide esters. They are hydrophobic surfactants for which the overall HLB is less than 9 (e.g., less than 6). Examples are SPAN (ICI Americas Inc; e.g., SPAN 80 or sorbitan monooleate), Dehymuls™ (Cognis; e.g., Dehymuls® SMO (sorbitan oleate)), Arlacel™ (ICI Americas Inc), and MONTANE™ (Seppic; e.g., MONTANE™ 80). Useful mannide esters include, for example, mannide monooleate (e.g., Sigma; or by MONTANIDE™ 80 by Seppic).

The O/W (e.g., squalene-based) emulsion adjuvant has an aqueous phase comprising water and, in some embodiments, a salt. The aqueous phase may, for example, be a buffered solution containing phosphate, acetate, citrate, succinate, or histidine. The buffered solution may have a pH of between about 6.4 and about 9 (e.g., pH of about 6.8 to about 7.5 such as 7.0, 7.2, or 7.4).

In some embodiments, the O/W (e.g., squalene-based) emulsion adjuvant may comprise toll-like receptor (TLR) agonists (e.g., TLR4 agonist ER804057 or E6020), polyols (e.g., sorbitol, mannitol, glycerol, xylitol or erythritol), and/or mineral salts (e.g., aluminum salts such as aluminum hydroxide, aluminum potassium sulfates, and aluminum phosphate; calcium salts; or iron salts).

The O/W (e.g., squalene-based) emulsion adjuvant may be prepared through a phase-inversion-temperature (PIT) process that leads to a monodisperse emulsion, the droplet size of which is small (e.g., submicron), making the emulsion highly stable and readily filterable by means of sterilizing filters. This process comprises a step in which a W/O inverse emulsion is obtained by raising the temperature and a step in which the W/O inverse emulsion is converted to an O/W emulsion by lowering the temperature. This conversion takes place when the W/O emulsion obtained is cooled to a temperature below the phase inversion temperature of this emulsion. The O/W emulsion made by this process is considered “thermo-reversible.”

In general, the thermo-reversible emulsion used herein is homogeneous. The term “homogeneous emulsion” refers to an emulsion for which the graphic representation of size distribution (“granulogram”) of the oil droplets is unimodal. Typically, this graphic representation is of the “Gaussian” type. In some embodiments, at least 90% of the population by volume of the oil droplets of the emulsion has a size no greater than 200 nm (e.g., 50-200 nM, 75-175 nM, 75 to 150 nM, 75-125 nM, 75-100 nM, 80-120 nM, or 90-110 nM). In general, at least 50% (e.g., at least 60, 65, 70, 75, 80, 85, 90, or 95%) of the population by volume of the oil droplets of these emulsions has a size no greater than 110 nm. According to one specific characteristic, at least 90% of the population by volume of the oil droplets has a size no greater than 180 nm and at least 50% (e.g., at least 60, 65, 70, 75, 80, 85, 90, or 95%) of the population by volume of the oil droplets has a size no greater than 110 nm. The size of the droplets can be measured by various means, e.g., laser diffraction particle size analyzers such as the Beckman Coulter devices of the LS range (e.g., the LS230) or Malvern devices of the Mastersizer range (e.g., Mastersizer 2000).

In certain embodiments, the adjuvant is the AF03 adjuvant. AF03 is a squalene-based O/W emulsion (Klucker et al., J Pharm Sci. (2012) 101(12):4490-500; Rudicell et al., Vaccine (2019) 37(42):6208-20; Ruat et al., J Virol. (2008) 82(5):2565-9). For each 0.25 mL of AF03, the adjuvant contains 12.5 mg squalene, 1.85 mg sorbitan monooleate (e.g., Dehymuls SMO™), 2.38 mg POE (12) cetostearyl ether (e.g., Kolliphor CS12™), 2.31 mg mannitol, made up to a volume of 0.5 mL with phosphate-buffered saline (PBS) (7.5 mM phosphate, 150 mM NaCl; pH 7.2). See also U.S. Pat. No. 8,703,095 and WO2007/006939. AF03 is obtainable by a PIT process and has an average droplet size of about 100 nM, or more than 60% (e.g., about 85%) of its droplets are no bigger than 100 nm. In some embodiments, a single dose of AF03 for intramuscular injection (e.g., for adult humans) is 0.25 mL. See also Table 9, infra. The single dose of AF03 may be mixed with a single dose of an antigen component provided in the same liquid volume, to reach a final volume of 0.5 mL for, e.g., intramuscular injection.

A potential safety issue with coronavirus vaccines is the ability to potentiate immunopathology from vaccines upon exposure to wild-type virus (Smatti et al., Front Microbiol. (2018) 9:2991). The molecular mechanism for this phenomenon, termed antibody-dependent enhancement or immune enhancement of viral infection, is still not fully understood. In the context of coronavirus infections, various factors have been suggested as potentially contributing to the phenomenon. These include the epitope targeted, the method of delivery of the antigen, the magnitude of the immune responses, the balance between binding and functional antibodies, the elicitation of antibodies with functional characteristics such as binding to particular Fc receptors, and the nature of the T-helper cell response (Tseng et al., PLoS One (2012) 7(4); Yasui et al., J Immunol. (2008) 181(9):6337-48; Czub et al., Vaccine (2005) 23(17-18):2273-9). The inclusion of adjuvanted formulations including adjuvants such as AF03 is anticipated to further enhance the magnitude of neutralizing antibody responses and thereby alleviate antibody-dependent enhancement of viral infection, which is thought to be mediated mostly by non-neutralizing antibodies.

### III. Production of Recombinant S Protein

The viral antigen component of the present immunogenic compositions may be produced by recombinant technology in insect cells (e.g., Drosophila S2 cells, Spodoptera frugiperda cells, Sf9 cells, Sf21, High Five cells, or expresSF+ cells) that have been transduced with a baculoviral expression vector, such as one derived from Autographa californica multiple nucleopolyhedrovirus (AcMNPV). Baculoviruses such as AcMNPV form large protein crystalline occlusions within the nucleus of infected cells, with a single polypeptide termed polyhedrin accounting for approximately 95% of the protein mass. The gene for polyhedrin is present as a single copy in the baculoviral genome and can be readily replaced with foreign genes because it is not essential for virus replication in cultured cells. Recombinant baculoviruses that express a foreign gene such as the recombinant S polypeptide are constructed by way of homologous recombination between baculovirus genomic DNA and a transfer plasmid containing the foreign gene.

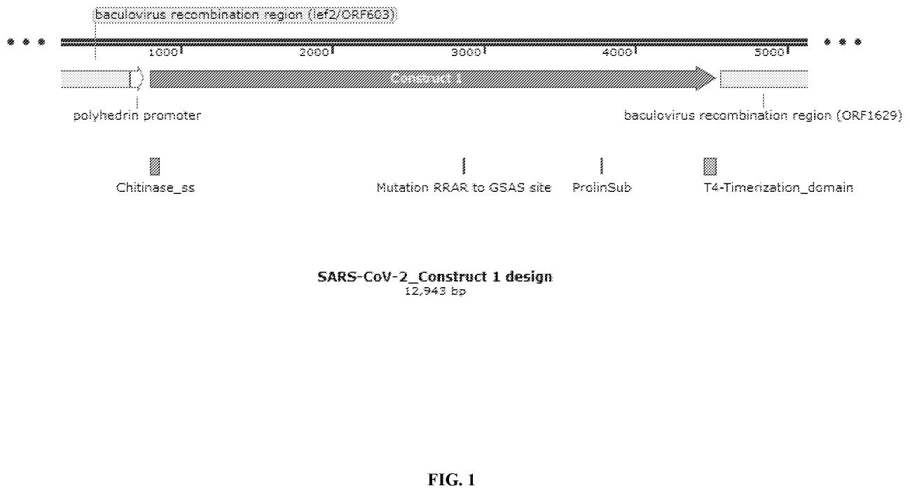

In certain embodiments, the transfer plasmid contains an expression cassette for the recombinant S polypeptide, where the expression cassette is flanked by sequences naturally flanking the polyhedrin locus in the AcMNPV (FIG. 1). The transfer plasmid is co-transfected into host cells with baculovirus genomic DNA that has been linearized with an enzyme (e.g., Bsu36I) that removes the polyhedrin gene and a part of an essential gene downstream of the polyhedrin locus so that parental viral DNA molecule cannot replicate, rendering the genomic DNA non-infectious; however, this part of the essential gene is present on the transfer plasmid. After co-transfection, homologous recombination between the transfer plasmid and the linearized genomic DNA recircularizes the genomic viral DNA, restoring its ability to replicate. Because the original baculovirus genomic DNA before linearization contains the polyhedrin gene, plaques formed by non-recombinant virus are cloudy (due to the crystalline occlusions in the infected cells), whereas plaques formed by recombinant virus are clear.

The baculoviral expression vector may be engineered to increase the yield of the recombinant protein. In some embodiments, the baculoviral vector has one or more genes knocked out. The baculovirus genome contains genes that are non-essential for virus replication in cell culture and for expression of recombinant proteins. Deletion of such genes may remove unnecessary genetic burden, help generate more stable baculoviral expression vectors, reduce time needed for established insect cell infection, and result in more efficient expression of the recombinant protein. In some embodiments, the polyhedrin promoter is modified by including in it more than one copy of the burst sequence; for example, the promoter may be engineered to include two burst sequences to create a “double burst” (DB) promoter, which contains two repeats of the nucleotide sequence CTGTTTTCGTAACAGTTTTGTAATAAAAAAACCTATAAATA (SEQ ID NO:12). See, e.g., Manohar et al., Biotechnol Bioeng. (2010) 107:909-16. To integrate the viral antigen coding sequence into a baculoviral expression vector, a transfer plasmid carrying the coding sequence may be integrated to the DNA encoding the baculoviral genome through homologous recombination. Viral identity may be confirmed by, for example, Southern blot or Sanger sequencing analysis of the S protein coding sequence insert from purified baculovirus DNA and Western blot analyses of the recombinant protein produced in infected insect cells. See, e.g., U.S. Pat. Nos. 6,245,532 and 8,541,003.

Host cells containing the viral antigen expression construct are cultured in bioreactors (e.g., 45 L, 60 L, 459 L, 2000 L, or 20,000 L) in, e.g., a batch process or a fed-batch process. The produced S protein may be isolated from the cell cultures by, for example, column chromatography in either flow-through or bind-and-elute modes. Examples are ion exchange resins and affinity resins, such as lentil lectin Sepharose, and mixed mode cation exchange-hydrophobic interaction columns (CEX-HIC). The protein may be concentrated, buffer exchanged by ultrafiltration, and the retentate from the ultrafiltration may be filtered through a 0.22 μm filter. See, e.g., McPherson et al., “Development of a SARS Coronavirus Vaccine from Recombinant Spike Protein Plus Delta Inulin Adjuvant,” Chapter 4, in Sunil Thomas (ed.), Vaccine Design: Methods and Protocols: Volume 1: Vaccines for Human Diseases, Methods in Molecular Biology, Springer, New York, 2016. See also U.S. Pat. No. 5,762,939.

The baculovirus expression vector system (BEVS) provides an excellent method for the development of the ideal subunit vaccine. Recombinant protein can be produced by such systems in approximately eight weeks. Speedy production is especially critical when there is a pandemic threat. Further, baculoviruses are safe by virtue of their narrow host range, which is restricted to a few taxonomically related insect species, and have not been observed to replicate in mammalian cells. Additionally, very few microorganisms are known to be able to replicate in both insect cells and mammalian cells; thus, the possibility of adventitious agent contamination in clinical products made by insect cells is very low. Moreover, humans generally do not have pre-existing immunity to proteins from insects that are the natural hosts for baculoviruses, because these insects are non-biting; thus, allergic reactions to clinical products made in BEV systems are not likely. Further, although the carbohydrate moieties added to proteins in insect cells appear to be less complex than those on their mammalian cell-expressed counterparts, the immunogenicity of insect cell-expressed and mammalian cell-expressed glycoproteins appear to be equivalent. Full-length proteins expressed in baculovirus systems usually self-assemble into the higher-order structures normally assumed by the natural proteins by modulating the surfactant concentration. Finally, the BEVS system is highly efficient due to the extremely high activity of the polyhedrin promoter, which allows production of recombinant protein at high levels at significantly lower costs.

### IV. Formulation and Packaging of Vaccines

The recombinant S protein(s) (e.g., preS dTM) can be formulated and packaged, alone or in combination with an adjuvant in an amount effective to enhance the immunogenic response against the recombinant S protein. The immunogenic composition may be monovalent or multivalent as described above. The immunogenic composition may be formulated for parenteral (e.g., intramuscular, intradermal or subcutaneous) administration or nasopharyngeal (e.g., intranasal) administration. The composition may be with or without a pharmaceutically acceptable preservative. Such preservatives include, without limitation, parabens, thimerosal, thiomersal, chlorobutanol, benzalkonium chloride, and chelators (e.g., EDTA).

The immunogenic composition may be provided in the form of a mixture of the antigen with an adjuvant, provided that the adjuvant does not comprise both tocopherol and squalene or the AS03 adjuvant.

The immunogenic composition may also be in the form of an extemporaneous formulation, where the antigen and the adjuvant are brought into contact just before or at the time of use. For example, the antigen (liquid) can be mixed volume to volume with the adjuvant (emulsion) prior to injection. In some embodiments, the antigen formulation, prior to mixing with the adjuvant, is an aqueous buffered solution. The buffer may be a phosphate buffered saline, optionally prepared with monobasic sodium phosphate, dibasic sodium phosphate, and sodium polysorbate. The buffer may also comprise a surfactant (e.g., at 0.01-10%).

In some embodiments, the surfactant is hydrophilic and/or nonionic. The surfactant can be selected from: ethoxylated polysorbates, such as polysorbate 20, polysorbate 40, polysorbate 60 and polysorbate 80 marketed respectively under the brand names Tween® 20, Tween® 40, Tween® 60, and Tween® 80; ethylene oxide/propylene oxide copolymers, called poloxamers hereinafter, such as poloxamer 124 marketed under the brand name Synperonic™ PE/L44, poloxamer 188 marketed under the brand name Pluronic® F68 or Synperonic™ PE/F68, poloxamer 237 marketed under the brand name Pluronic® F87 or Synperonic™ PE/F87; poloxamer 338 marketed under the brand name Synperonic™ PE/F108, or poloxamer 407 marketed under the brand name Pluronic® F127, Synperonic™ PE/F127, or Lutrol® F127; and polyethylene hydroxystearates, such as polyethylene hydroxystearate 660 marketed under the brand name Kolliphor® HS 15.

In some embodiments, the aqueous buffered formulation containing the antigen may comprise 0.01-0.5% polysorbate 20. In some embodiments, the formulation contains about 0.02% to 0.2% polysorbate 20. In certain embodiments, for every 0.25 mL of aqueous antigen formulation (without adjuvant), the formulation contains 50-600 (e.g., 55 or 550) μg polysorbate 20.

In some embodiments, the antigen can be lyophilized and taken up with the adjuvant (emulsion) just before use or, conversely, the adjuvant can be in a lyophilized form and taken up with a solution (e.g., an aqueous buffered solution) of the antigen.

Accordingly, the present disclosure provides an article of manufacture, such as a kit, that provides the antigen and adjuvant components of the present immunogenic composition in separate containers (e.g., pre-treated glass vials or ampules), and the two components are mixed prior to injection. If a solution is needed for resuspension of a lyophilized component, that solution may be provided in the article of manufacture such as a kit as well. Alternatively, the antigen component and the adjuvant are mixed and provided in the same container, and the composition can be administered directly to subjects in need of vaccination. The article of manufacture may include instructions for use as well. The article of manufacture (e.g., the kit) may also include instructions for use.

The immunogenic composition may be provided in a unit dosage format (single dose), or in a multi-dose format. In some embodiments, the antigen component is provided in a multi-dose format in one container and the adjuvant component is provided in a single- or multi-dose format in a separate container; prior to use, a single dose of the antigen component is taken from its container and mixed with a single dose of the adjuvant.

In some embodiments, the immunogenic composition is provided for use in intramuscular (IM) or subcutaneous injection. The immunogenic composition, once made up at bedside by mixing the antigen component and the adjuvant component, can be injected to a subject at, e.g., his/her deltoid muscle in the upper arm. In some embodiments, the antigen and/or adjuvant components of the immunogenic composition is provided in a pre-filled syringe or injector (e.g., single-chambered or multi-chambered). In some embodiments, the immunogenic composition is provided for use in inhalation and is provided in a pre-filled pump, aerosolizer, or inhaler.

In some embodiments, the unit dosage for IM injection is 1-50 or 5-50 (e.g., 2.5, 5, 10, 15, 30, or 45) μg per dose of recombinant S protein (e.g., one or more, such as two, recombinant S proteins selected from preS dTM and variants thereof) in, e.g., an injection volume of about 0.2 to 0.6 mL (e.g., 0.25 mL or 0.5 mL). In some embodiments, the unit dosage is a total of 2.5 μg recombinant S protein in a 0.25 or 0.5 mL injection volume. In other embodiments, the unit dosage is a total of 5 μg recombinant S protein in a 0.25 or 0.5 mL injection volume. In some other embodiments, the unit dosage is a total of 10 μg recombinant S protein in a 0.25 or 0.5 mL injection volume. In some other embodiments, the unit dosage is a total of 15 μg recombinant S protein in a 0.25 or 0.5 mL injection volume. In some embodiments, the unit dosage is a total of 45 μg recombinant S protein in a 0.25 or 0.5 mL injection volume. In these embodiments, the 0.25 mL or 0.5 mL injection volume may include an adjuvant.

In some embodiments, the recombinant S protein is supplied in a container in a single dose or multiple doses. Each dose may be in a volume of, e.g., 0.25 mL. The S protein may be formulated in a phosphate buffered saline (q.s. 0.25 mL) with a concentration of 0.2% Tween 20® without preservatives or antibiotics. The protein solution may be mixed with an adjuvant (e.g., an AF03 adjuvant; not an AS03 adjuvant) prior to use. In some embodiments, the protein solution is mixed with an equal volume of the adjuvant prior to use.

In some embodiments, one unit dosage of an antigen composition for IM injection contains the ingredients as shown in Table A below.

TABLE A
CoV2 preS dTM Formulation (Non-Adjuvanted)

| Name of drug ingredient/substance | Quantity per dose | |
|---|---|---|
| Recombinant preS dTM protein | 2-50 | μg |
| Sodium phosphate monobasic monohydrate | 0.097 | mg |
| Sodium phosphate dibasic anhydrous | 0.26 | mg |
| Sodium chloride | 2.2 | mg |
| Polysorbate 20 (Tween 20 ®) | 0.55 | mg |

| | |
|---|---|
| Water for injection | q.s. final 0.25 mL |

In some embodiments, this unit dosage includes 2.5, 5, or 10 μg of D614 preS dTM (SEQ ID NO:10, without the signal sequence). In some embodiments, this unit dose includes 2.5, 5, or 10 μg of B.1.351 preS dTM (SEQ ID NO:14, without the signal sequence). In some embodiments, this unit dosage is bivalent and includes a total of 2.5, 5, or 10 μg of D614 preS dTM and B.1.351 preS dTM, where the two proteins exist in equal amounts. The unit dosage of antigen composition may be used alone for vaccination, or mixed with an adjuvant prior to vaccination.

In some embodiments, the unit dosage is a total of 2.5, 5, 10, 15, or 45 μg recombinant S protein in 0.25 mL or 0.5 mL, not including adjuvant. The dose may be administered, for example, as a booster dose, as further explained below, with or without an adjuvant.

In some embodiments, for each human vaccination by IM injection, 2.5 μg preS dTM (SEQ ID NO:10, without the signal sequence) or a variant (e.g., SEQ ID NO:14, without the signal sequence) in 0.25 mL of a sterile, clear and colorless PBS solution (see, e.g., Table A, Table 8 or Table 8A below) is mixed volume to volume with 0.25 mL of an AF03 adjuvant prior to injection, to reach a final injection volume of 0.5 mL. In other embodiments, this antigen solution is administered as a booster, without an adjuvant or with another adjuvant, provided that the adjuvant does not comprise both tocopherol and squalene, or AS03.

In some embodiments, for each human vaccination by IM injection, 5 μg preS dTM (SEQ ID NO:10, without the signal sequence) or a variant (e.g., SEQ ID NO:14, without the signal sequence) in 0.25 mL of a sterile, clear and colorless PBS solution (see, e.g., Table A, Table 8 or Table 8A, infra) is mixed volume to volume with 0.25 mL of AF03 prior to injection, to reach a final injection volume of 0.5 mL. In other embodiments, this antigen solution is administered as a booster, without an adjuvant or with another adjuvant, provided that the adjuvant does not comprise both tocopherol and squalene, or AS03.

In some embodiments, for each human vaccination by IM injection, 10 μg preS dTM (SEQ ID NO:10, without the signal sequence) or a variant (e.g., SEQ ID NO:14, without the signal sequence) in 0.25 mL of a sterile, clear and colorless PBS solution (see, e.g., Table A, Table 8 or Table 8A, infra) is mixed volume to volume with 0.25 mL of AF03 prior to injection, to reach a final injection volume of 0.5 mL. In other embodiments, this antigen solution is administered, as a booster, without an adjuvant or with another adjuvant, provided that the adjuvant does not comprise both tocopherol and squalene, or AS03.

In some embodiments, for each human vaccination by IM injection, 15 μg preS dTM (SEQ ID NO:10, without the signal sequence) or a variant (e.g., SEQ ID NO:14, without the signal sequence) in 0.25 mL of a sterile, clear and colorless PBS solution (see, e.g., Table A, Table 8 or Table 8A, infra) is mixed volume to volume with 0.25 mL of AF03 prior to injection, to reach a final injection volume of 0.5 mL. In other embodiments, this antigen solution is administered without an adjuvant or with another adjuvant, provided that the adjuvant does not comprise both tocopherol and squalene, or AS03.

In some embodiments, for each human vaccination by IM injection, 45 μg preS dTM (SEQ ID NO:10, without the signal sequence) or a variant (e.g., SEQ ID NO:14, without the signal sequence) in 0.25 mL of a sterile, clear and colorless PBS solution (see, e.g., Table A, Table 8 or Table 8A, infra) is mixed volume to volume with 0.25 mL of AF03 prior to injection, to reach a final injection volume of 0.5 mL. In other embodiments, this antigen solution is administered without an adjuvant or with another adjuvant, provided that the adjuvant does not comprise both tocopherol and squalene, or AS03.

In some embodiments, for each human vaccination by IM injection, a total of 10 μg of two different recombinant S protein (e.g., preS dTM or a variant such as one derived from B.1.351 (SEQ ID NO:14, without the signal sequence), 5 μg each) in 0.25 mL of a sterile, clear and colorless PBS solution (see, e.g., Table A, Table 8 or Table 8A below) is mixed volume to volume with 0.25 mL of an AF03 adjuvant prior to injection, to reach a final injection volume of 0.5 mL.

In some embodiments, the immunogenic composition is monovalent and contains 10 μg per dose of a single recombinant S protein (e.g., preS dTM or a preS dTM variant such as B.1.351 preS dTM).

In some embodiments, the immunogenic composition is bivalent and contains two different recombinant S proteins (e.g., preS dTM and a preS dTM variant such as B.1.351 preS dTM) at 5 μg each per dose.

In some embodiments, the immunogenic composition is trivalent and contains three different recombinant S proteins (e.g., preS dTM and two different preS dTM variants) at 3.3 μg each per dose.

In some embodiments, the immunogenic composition is monovalent and contains a single recombinant S protein (e.g., preS dTM or a preS dTM variant such as B.1.351 preS dTM) at 2.5 μg per dose.

In some embodiments, the vaccine product of the present disclosure may be stored at 2-8° C.

### V. Use of the Vaccines

Subjects suitable for vaccination by the vaccine compositions of the present disclosure include humans susceptible for SARS-CoV-2 infections, such as adults 18-49 years old, 18-59 years old, adults 50 years or older, adults 60 years or older, adults 65 years or older, children 2-18 years old, children under 12 years of age, or children under 2 years of age. The amount of vaccine to be administered to the subjects can be determined in accordance with standard techniques well known to those of ordinary skill in the art, including the type of adjuvant used, the route of administration, and the age and weight of the subject. In some embodiments a 2.5 μg dose of antigen, with or without adjuvant, will be administered. In some embodiments a 5 μg dose of antigen, with or without adjuvant, will be administered. In some embodiments, a 10 μg dose of antigen, with or without adjuvant, will be administered. In some embodiments a 15 μg dose of antigen, with or without adjuvant, will be administered. In some embodiments a 45 μg dose of antigen, with or without adjuvant, will be administered. The compositions may be administered in a single dose or in a series of doses (e.g., one to three primary doses with subsequent “booster” dose(s)). In some embodiments a first and second dose will be administered about 14 days (or about 2 weeks) to about six months apart. For example, the interval between doses may be 14-35 days (e.g., about 21 or 28 days) or about 2-5 weeks (e.g., about 3 or 4 weeks) apart.

In some embodiments, a single dose is a mixture of about 0.25 mL of an antigen composition as shown in Table A, Table 8 or Table 8A (containing 5 or 10 μg recombinant S protein) and an adjuvant (e.g., AF03). In further embodiments, a subject is given two such doses, each dose being 21 days or 3 weeks part. In other further embodiments, a subject is given two such doses, each dose being 28 days or 4 weeks part.

The vaccine composition is provided to the subject in a prophylactically effective amount, which may be administered in a single dose or in a series of doses. A “prophylactically effective amount” refers to the amount required to induce an immune response sufficient to prevent or delay onset, and/or reduce in frequency and/or severity, of one or more symptoms of COVID-19. In some embodiments, the amount elicits an immune response that reduces partially or completely the severity of one or more symptoms and/or time over which one or more symptoms are experienced by the subject, reduces the likelihood of developing an established infection after challenge, slows progression of illness, optionally extending survival, and/or produces neutralizing antibodies to SARS-CoV-2 and a SARS-CoV-2 S protein specific T cell response.

In some embodiments, the vaccination method provided herein prevents or ameliorates COVID-19, such as one or more of its symptoms, or prevents or reduces the risk of hospitalization or death associated with COVID-19. In one method, a COVID-19 naïve or unvaccinated subject is administered by IM an immunogenic composition prepared by mixing 0.25 mL of an aqueous antigen component and an adjuvant. The 0.25 mL aqueous antigen component may be monovalent (MV) and comprises 5 or 10 μg of D614 preS dTM or B.1.351 (Beta) preS dTM, optionally formulated in PBS as shown in Table A. Alternatively, the aqueous antigen component is bivalent (BV) and comprises 5 μg of D614 preS dTM and 5 μg of Beta preS dTM, optionally formulated in PBS as shown in Table A; or comprises 2.5 μg of D614 preS dTM and 2.5 μg of Beta preS dTM, optionally formulated in PBS as shown in Table A. The subject may be administered with the immunogenic composition twice, three weeks or four weeks apart, or one month apart.

### VI. Use of the Vaccines as Boosters

The present vaccine compositions may be used as a universal booster. The present vaccine compositions may be used as boosters for previously administered COVID-19 vaccines, as part of a prime-boost vaccination regimen, e.g., as heterologous or homologous prime-boost vaccination regimen. The prime doses in the regimen (i.e., primary vaccines) may be vaccines that are based on mRNAs, DNAs, viral vectors (e.g., adenoviral vectors, adeno-associated viral vectors, lentiviral vectors, vesicular stomatitis viral vectors, vaccinia viral vectors, or measles viral vectors), peptides or proteins, viral-like particles (VLP), capsid-like particles (CLP), live attenuated viruses, inactivated viruses (killed vaccines), and the like. In some embodiments, the primary vaccine contains the same antigen as the booster vaccine (i.e., a homologous prime-boost vaccination regimen). A prime-boost regimen may be advantageous in part due to re-utilization, especially for a viral vector prime and to a qualitatively and quantitatively different immune profile provided by the boost. Such regimens are expected to lead to an enhanced outcome in terms of breadth, potency, and durability of the anti-viral immunity in vaccinated subjects.

Vaccines comprising genetic materials (e.g., mRNAs, DNAs, or viral vectors) for expressing a SARS-CoV-2 antigen (e.g., an S protein antigen) in the body are collectively called “genetic vaccines.” For examples, genetic vaccines include those comprising mRNA, with or without chemical modifications or nucleotide analogs. The mRNA may be encapsulated (e.g., in lipid nanoparticles (LNP)) or complexed with a carrier or adjuvant (e.g., protamine or saponin). The mRNA may be self-replicating or non-self-replicating. The present vaccine compositions are useful as boosters for genetic vaccines, because genetic vaccines could elicit in the vaccinated subjects an anti-drug immune response that destroys and therefore reduces the efficacy of subsequent doses of the same vaccines. In such instances, the genetic vaccines cannot be administered to the same subjects repeatedly (e.g., seasonally).

In some embodiments of the present prime-boost regimen, the prime doses may be genetic vaccines encoding a recombinant S protein, which may include an ectodomain of the SARS-CoV-2 S protein. In some embodiments, the recombinant S protein may comprise an amino acid sequence in SEQ ID NO:1, 4, 10, 13, or 14, or an antigenic fragment therein. In some embodiments, the recombinant S protein is a trimer of a polypeptide comprising a sequence from an SARV-CoV-2 ectodomain or receptor-binding domain (RBD) and a trimerization sequence (e.g., the native SARS-CoV-2 S trimerization domain). In some embodiments, the encoded recombinant S protein may comprise a signal peptide sequence (e.g., a signal peptide from SARS-CoV-2 such as the S protein) that facilitates the secretion of the recombinant S protein from the producing cells in the vaccinated subject.

In some embodiments, the genetic vaccine encodes an S protein or an antigenic portion thereof that has one or more mutations as compared to a reference (e.g., naturally occurring) S protein for specific design purposes. For example, the encoded S protein may contain (i) mutations at the furin cleavage site to prevent furin cleavage (e.g., the “GSAS” (SEQ ID NO:6) mutations), (ii) mutations that alter endoplasmic reticulum (ER) retention, (iii) mutations that abrogate putative glycosylation, (iv) mutations that introduce an alternative signal peptide, and/or (v) mutations that stabilize the prefusion conformation of the S polypeptide (e.g., the “PP” mutations).

In some embodiments, the S protein encoded by the genetic vaccine may include naturally occurring mutations such as the D614G mutation and the other mutations described herein. In certain embodiments, the genetic vaccine may encode a recombinant S protein derived from a SARS-Cov-2 variant such as one described above.

In certain embodiments, the genetic vaccines, such as mRNA vaccines, may encode the following recombinant S polypeptide:

| (SEQ ID NO: 13) | |
|---|---|
| 1 | MFVFLVLLPL VSSQCVNLTT RTQLPPAYTN SFTRGVYYPD KVFRSSVLHS |
| 51 | TQDLFLPFFS NVTWFHAIHV SGTNGTKRFD NPVLPFNDGV YFASTEKSNI |
| 101 | IRGWIFGTTL DSKTQSLLIV NNATNVVIKV CEFQFCNDPF LGVYYHKNNK |
| 151 | SWMESEFRVY SSANNCTFEY VSQPFLMDLE GKQGNFKNLR EFVFKNIDGY |
| 201 | FKIYSKHTPI NLVRDLPQGF SALEPLVDLP IGINITRFQT LLALHRSYLT |
| 251 | PGDSSSGWTA GAAAYYVGYL QPRTFLLKYN ENGTITDAVD CALDPLSETK |
| 301 | CTLKSFTVEK GIYQTSNERV QPTESIVRFP NITNLCPFGE VFNATRFASV |
| 351 | YAWNRKRISN CVADYSVLYN SASFSTFKCY GVSPTKLNDL CFTNVYADSF |
| 401 | VIRGDEVRQI APGQTGKIAD YNYKLPDDFT GCVIAWNSNN LDSKVGGNYN |
| 451 | YLYRLFRKSN LKPFERDIST EIYQAGSTPC NGVEGFNCYF PLQSYGFQPT |
| 501 | NGVGYQPYRV VVLSFELLHA PATVCGPKKS TNLVKNKCVN FNFNGLTGTG |
| 551 | VLTESNKKEL PFQQFGRDIA DTTDAVRDPQ TLEILDITPC SFGGVSVITP |
| 601 | GTNTSNQVAV LYQDVNCTEV PVAIHADQLT PTWRVYSTGS NVFQTRAGCL |
| 651 | 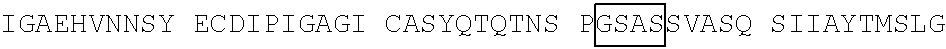 |
| 701 | AENSVAYSNN SIAIPTNFTI SVTTEILPVS MTKTSVDCTM YICGDSTECS |
| 751 | NLLLQYGSFC TOLNRALTGI AVEQDKNTQE VFAQVKQIYK TPPIKDFGGF |
| 801 | NFSQILPDPS KPSKRSFIED LLFNKVTLAD AGFIKQYGDC LGDIAARDLI |
| 851 | CAQKENGLTV LPPLLTDEMI AQYTSALLAG TITSGWTFGA GAALQIPFAM |
| 901 | QMAYRFNGIG VTQNVLYENQ KLIANQFNSA IGKIQDSLSS TASALGKLQD |
| 951 | VVNQNAQALN TLVKQLSSNF GAISSVLNDI LSRLDPPEAE VQIDRLITGR |
| 1001 | LQSLQTYVTQ QLIRAAEIRA SANLAATKMS ECVLGQSKRV DFCGKGYHLM |
| 1051 | SFPQSAPHGV VFLHVTYVPA QEKNFTTAPA ICHDGKAHFP REGVFVSNGT |
| 1101 | HWFVTQRNFY EPQIITTDNT FVSGNCDVVI GIVNNTVYDP LQPELDSFKE |
| 1151 | ELDKYFKNHT SPDVDLGDIS GINASVVNIQ KEIDRLNEVA KNLNESLIDL |
| 1201 | QELGKYEQYI KWPWYIWLGF IAGLIAIVMV TIMLCCMTSC CSCLKGCCSC |
| 1251 | GSCCKFDEDD SEPVLKGVKL HYT |

In the above sequence, the boxed sequence (GSAS; SEQ ID NO:6) is changed from the wildtype RRAR (SEQ ID NO:5). The underlined residues (PP) is changed from the wildtype KV. These changes may help maintain the trimeric S protein in a stable prefusion conformation.

In some embodiments, the genetic vaccine is Moderna COVID-19 Vaccine, Pfizer-BioNTech COVID-19 Vaccine, Janssen COVID-19 Vaccine, or Vaxzevria (formerly COVID-19 Vaccine AstraZeneca).

In some embodiments of the present prime-boost regimen, the prime doses are killed vaccines, such as Sinovac-CoronaVac and the Sinopharm BIBP vaccine.

The prime-boost regimen comprises vaccination with a primary vaccine (e.g., a genetic vaccine or a subunit vaccine) and then one or more booster doses with the present protein vaccine. In some embodiments, the primary vaccine entails one administration (e.g., intramuscular, subcutaneous, intradermal, or intranasal administration) of the vaccine, or two administrations of the vaccine separated by a period of time (e.g., about 2, 3, 4, 5, 6, 7, 8, 9, or 10 weeks, or longer).

In some embodiments, a booster dose with the present recombinant protein may be given at least two weeks (e.g., four weeks, one month, two months, three months, four months, five months, six months, seven months, eight months, nine months, ten months, eleven months, one year, one and a half years, two years, three year, four years, five years, or longer) after the primary vaccination. For example, once a genetic vaccine (e.g., an mRNA or adenoviral-based vaccine) or a subunit vaccine is administered, a booster dose with the present protein vaccine may be given to the subject annually or semi-annually. For convenience, the booster vaccine may be co-administered with a flu vaccine annually (e.g., as separate formulations or co-formulation).

In some embodiments, the booster is a monovalent or multivalent immunogenic composition described herein, used with or without an adjuvant. In some embodiments, the booster is a monovalent immunogenic composition (e.g., one containing a recombinant S protein derived from the Wuhan strain or the South African variant). In other embodiments, the booster is a bivalent immunogenic composition (e.g., one containing a recombinant S protein derived from the Wuhan strain and a recombinant S protein derived from the South African variant).

In certain embodiments, a booster dose may be a 0.25 or 0.5 mL immunogenic composition comprising 2.5 or 5 μg preS dTM or variant(s) thereof. In some embodiments, the booster shot does not include an adjuvant. In some embodiments, the booster dose contains an adjuvant (e.g., an AF03 adjuvant; not an AS03 adjuvant), and may, for example, be prepared by mixing a solution comprising the antigen volume to volume with an adjuvant prior to injection. In some embodiments, the booster shot is prepared by mixing, prior to injection, 2.5 or 5 μg of preS dTM or a variant in 0.25 mL of a sterile, clear and colorless PBS solution (see, e.g., Table A, Table 8 or Table 8A below) volume to volume with 0.25 mL of an AF03 adjuvant. In further embodiments, the variant is the Beta variant (e.g., SEQ ID NO:14 with the signal sequence).

In certain embodiments, the primary vaccination is carried out with a subunit vaccine comprising a recombinant S protein, and the booster vaccine contains a lower amount of a recombinant S protein than the vaccine used for the primary (non-booster) vaccination. For example, the primary vaccination entails two shots, with 10 μg recombinant S protein per shot, separately by an interval (e.g., an interval of 3, 4, 5, 6, 7, 8, or more weeks), whereas a booster shot may contain just 2.5 or 5 μg recombinant S protein.

In some embodiments, the primary vaccination entails two shots of a 0.5 mL immunogenic composition prepared by mixing, prior to injection, 10 μg of preS dTM or a variant (or 5 μg of preS dTM plus 5 μg of a variant, for a bivalent vaccine) in 0.25 mL of a sterile, clear and colorless PBS solution (see, e.g., Table A, Table 8 or Table 8A below) volume to volume with 0.25 mL of an AF03 adjuvant, with an interval (e.g., an interval of 3, 4, 5, 6, 7, 8, or more weeks) between the two shots. The subject is then given a booster vaccine at a later time (e.g., at least 3, 6, 8, 9, or 12 months after the second shot of the primary vaccination), wherein the booster vaccine may be 2.5 or 5 μg preS dTM or a variant in 0.25 or 0.5 mL of a sterile, clear and colorless PBS solution (see, e.g., Table A, Table 8 or Table 8A below), or may be prepared by mixing 2.5 or 5 μg preS dTM or a variant in 0.25 mL of the PBS solution volume to volume with 0.25 mL of an AF03 adjuvant.

In some embodiments, the booster vaccine does not require an adjuvant. The recombinant S protein may be provided in an aqueous liquid solution for IM injection (e.g., PBS, such as a PBS as shown in Table A, Table 8 or Table 8A).

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

In order that this invention may be better understood, the following examples are set forth. These examples are for purposes of illustration only and are not to be construed as limiting the scope of the invention in any manner.

### EXAMPLES

### Example 1: Cloning of SARS-CoV-2 S Encoding Sequences into Baculovirus Transfer Plasmids

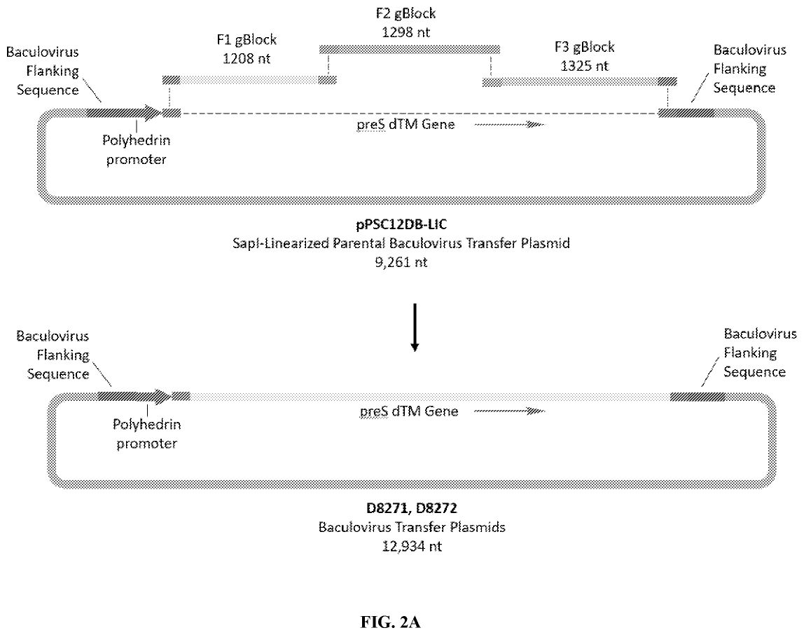

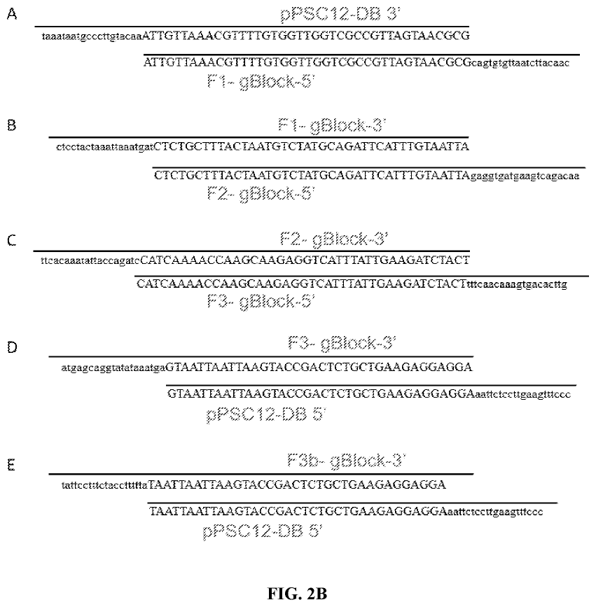

Gibson assembly (GA) was used to generate transfer plasmid harboring the indicated SARS-CoV-2 spike glycoprotein modified from SARS-CoV-2 spike glycoprotein, YP_009724390.1 from genome isolate Wuhan-Hu-1 GenBank NC045512. Three gene fragments (gBlocks) were designed for cloning into linearized SapI pPSC12 DB transfer vector, for each construct. The gBlock gene fragments have an overlapping 40 bp sequence at their junction sites and overlapping sequences with pPSC12 at 5′ and 3′ for gBlock fragment 1 and 3, respectively. gBlocks were synthesized by Integrated DNA Technologies (IDT). A depiction of the Gibson Assembly reaction is shown in (FIGS. 2A and 2B). Final transfer plasmid was confirmed via Sanger Sequencing by Eurofins Genomics. Site-directed mutagenesis may also be used to generate variant proteins.

### Example 2: Production and Purification of Recombinant S Protein

A recombinant baculovirus containing a sequence coding for preS dTM under the control of a polyhedrin promoter was used to infect S. frugiperda cells. Cells were grown at 27° C. to a density of 2.5×10⁶ cells/mL in PSFM medium (SAFC) and infected with 2% (volume/volume) of the recombinant baculovirus. Cells were harvested 72 hours post-infection by centrifugation for 15 minutes at 3,400×g. The supernatant was used for purification of recombinant S protein.

In one purification process, the supernatant containing the secreted recombinant SARS-CoV-2 Spike protein was depth-filtered using a SUPRACAP 100 dual layer K250P/KS50P 5″ filter (Pall, #NP5 LPDG41). The depth filtrate was concentrated 10× using 100 kDa Sartocon Slice Cassette, 0.1 m², flow rate of 200 mL/min at 15 psi followed by 5×diafiltration with 20 mM Tris; 50 mM NaCl, pH 7.4. The diafiltrate containing the SARS-CoV-2 Spike protein was purified by Capto™ Lentil Lectin (Cytiva) chromatography as the capture step purification. The Capto™ Lentil Lectin column was equilibrated with 20 mM Tris; 50 mM NaCl; 10 mM methyl-α-D-mannopyranoside, pH 7.4. Under these conditions, SARS-CoV-2 Spike protein binds to Capto™ Lentil Lectin resin and contaminants flowed through the column. The column was washed with 20 mM Tris; 50 mM NaCl; 10 mM methyl-α-D-mannopyranoside, pH 7.4 to remove unbound proteins. The SARS-CoV-2 Spike protein was eluted from the Capto™ Lentil Lectin column with elution buffer containing 20 mM Tris; 500 mM methyl-α-D-mannopyranoside, pH 7.4.

The Capto™ Lentil Lectin Eluate was further purified through Phenyl Sepharose™ HP Hydrophobic Interaction Chromatography resin (Cytiva) as the polishing step. The Capto™ Lentil Lectin eluate was adjusted to 750 mM ammonium sulfate concentration, 0.01% Triton X-100 concentration and loaded onto a Phenyl Sepharose HP column equilibrated with buffer containing 50 mM sodium phosphate; 750 mM ammonium sulfate; 0.01% v/v Triton X-100, pH 7.0. After loading, the Phenyl Sepharose HP column was washed with 50 mM sodium phosphate; 750 mM ammonium sulfate; 0.01% v/v Triton X-100, pH 7.0 to remove unbound contaminants. The SARS-CoV2 Spike protein was eluted from the Phenyl Sepharose HP column with elution buffer containing 50 mM sodium phosphate; 300 mM ammonium sulfate; 0.01% v/v Triton X-100, pH 7.0.

The Phenyl Sepharose HP Eluate was diluted 3.25× with distilled water and Q membrane filtration was performed using a single Mustang Q XT Acrodisc filter (Pall, #MSTGXT25Q16). Following the Q membrane filtration, TFF was performed using a Sartocon Slice 50 (Sartorius Stedim, #3D91465050ELLPU). The Q Filtrate was concentrated to 0.25 mg/mL and then diafiltered 10× with 10 mM sodium phosphate buffer, pH 6.8-7.2. The TFF retentate containing the SARS-CoV-2 Spike protein was formulated with 0.005% Tween 20 and sterile filtered using 0.2 μm filter and stored at 4° C. until use.

An alternative purification process uses CEX-HIC. Harvest may be accomplished with depth filtration (with or without an initial centrifugation step). Captured recombinant protein may then be further purified through ultrafiltration/diafiltration steps.

### Example 3: Pivotal Mouse Study

This example describes a study of a SARS-CoV-2 recombinant protein vaccine formulation in mice. The vaccine formulation contained a SARS-CoV-2 prefusion-stabilized S protein deleted for the transmembrane and cytoplasmic regions (CoV-2 preS dTM). The vaccine contained the AF03 adjuvant. This vaccine study investigated the dose response and adjuvant effect on humoral and cell-mediated immunity. The study also compared the effect between a non-stabilized S ectodomain (deleted for transmembrane and cytoplasmic region; “S dTM”) and preS dTM. S dTM contains SARS-CoV-2 spike protein ECD S1 and S2 regions, with a His tag (Sino Biological).

The mice used here were outbred female Swiss Webster mice, 6-8 weeks old. They were injected intramuscularly with 50 μL (25 μL antigen solution plus 25 μL adjuvant) of the vaccine formulation on Day 0 and Day 21.

The data below reflects targeted and actual antigen doses. After the experiment was run, a key polyclonal antibody reagent used to detect the SARS-CoV-2 preS protein was found to also recognize glycosylated host cell proteins (HCP). As a result, the purity and HCP levels targeted were inaccurate and the concentration of SARS-CoV-2 preS protein in the formulated vaccine product was significantly lower than planned. Table 1 shows the dosing regimen and Table 2 reflects the actual dose upon re-calculation as follows.

TABLE 1
Dosing Regimen for Mouse Study

| Group | Targeted Antigen | Adjuvant |
|---|---|---|
| (n = 10) | Dose (μg) | Dose (μg) |
| 1 | S dTM (4.5) | — |
| 2 | preS dTM (4.5) | AF03 (1,250) |
| 3 | preS dTM (1.5) | AF03 (1,250) |
| 4 | preS dTM (0.5) | AF03 (1,250) |
| 5 | preS dTM (0.167) | AF03 (1,250) |
| 6 | preS dTM (4.5) | — |
| 7 | preS dTM (1.5) | — |
| 8 | preS dTM (0.5) | — |
| 9 | preS dTM (0.167) | — |
| 10 | Diluent | — |

TABLE 2
CoV2-02_Ms Targeted and Actual
Doses of CoV2 preS dTM Antigen

| Injection | | CoV2 preS dTM | Actual |
|---|---|---|---|
| day | Targeted doses (μg) | content %* | doses (μg) |
| D 0 | 0.167/0.5/1.5/4.5 | 41 | 0.07/0.2/0.6/1.8 |
| D 21 | 0.167/0.5/1.5/4.5 | 26 | 0.04/0.13/0.4/1.17 |

*Actual doses were re-calculated based on new assays which differentiate structurally correct CoV2 preS dTM trimers from HCP impurities. The assays are based on ACE2 binding and/or HPSEC.

Because of the dosing adjustment based on new quantification assays, the actual doses were different for the D0 and D21 injections. For consistency, only the targeted doses are indicated in the text and figures.

Blood was drawn from the animals on Day −4, Day 21, and D 36. S-specific IgG, IgG₁, and IgG₂ₐ levels were measured by ELISA, where the plates were coated with spike ECD containing the S1 and S2 regions (S dTM; Sino Biological). Titers are reported as the inverse of the last dilution eliciting OD values greater than 0.2. OD=0.2 value represents at least two times higher than the assay background. The ability of the serum antibodies to neutralize live virus was assessed first in a plaque reduction neutralization test (PRNT) at BSL 3 using SARS-CoV-2 USA/WA1/2020 strain of the virus. A second neutralization assay was performed in parallel at BSL2 using the Integral Molecular SARS-CoV-2 GFP-pseudovirus assay on 293-hsACE2 clonal cells.

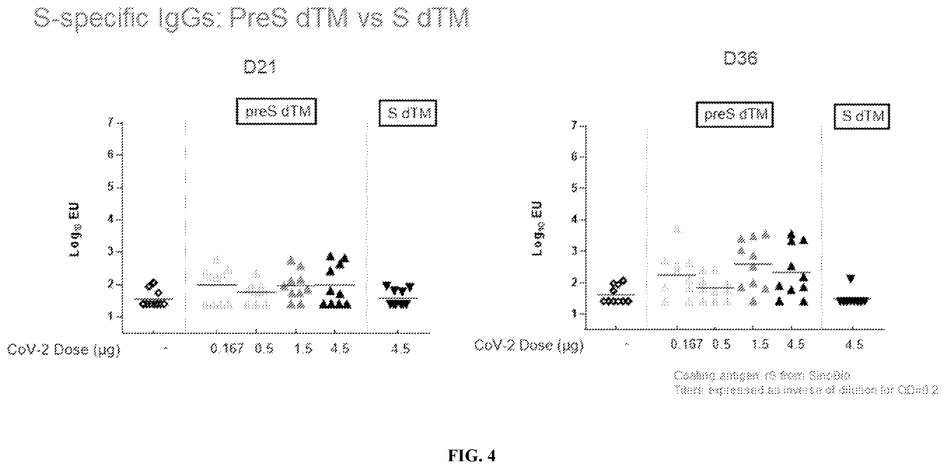

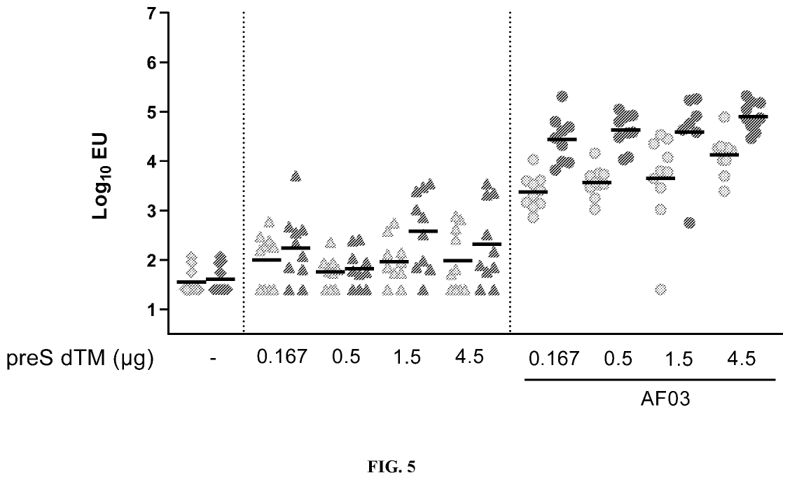

The data show that without adjuvant, preS dTM and S dTM were not immunogenic, as demonstrated by very low or absent IgG and neutralizing antibody responses after 1 or 2 doses. The serum S-specific IgG levels were similar between the two antigens and there was no statistically significant titer change from Day 21 to Day 36 (FIG. 4). By contrast, AF03-adjuvanted preS dTM vaccine elicited high IgG responses after 1 dose (D21), across all doses tested (mean ranged from 3.4 to 4.1 Log₁₀ ELISA Unit (EU) in the different vaccine dose group). The responses were further increased by the second injection (D36), and IgG mean titer reached 4.4 to 4.9 Log₁₀ EU depending on the vaccine dose. Both the adjuvant effect (fold increase and P-value) and a booster effect were demonstrated. Adjuvant AF03 significantly increased S-specific IgG titers in animals on both Day 21 and Day 36 induced by immunization with preS dTM, and Day 36 saw higher titers than Day 21 (FIG. 5). In summary, the dose-response effect of the AF03-containing vaccine formulation was statistically significant, with p<0.001. However, the dose-response effect of the non-adjuvanted formulation was not statistically significant (p=0.7866). In short, whatever dose was used, a significant AF03 adjuvant effect was shown, with all dosages having p values of <0.001.

Consistent with the IgG responses, the AF03-adjuvanted vaccine elicited robust neutralizing antibody responses after 2 doses, as evaluated in a PRNT assay. To perform this assay, serum samples were heat inactivated at 56° C. for 30 minutes and diluted in diluent (DMEM/2% FBS). SARS-CoV-2 virus was prepared and kept on ice until use. Diluted serum samples were mixed with an equal volume of SARS-CoV-2 diluted to contain 30 PFU per well and incubated for 1 hour at 37° C. Plates of confluent Vero E6 cells were inoculated with 250 μL of the serum+virus mixtures in duplicates and incubated at 37° C. for 1 h. After incubation, plates were overlaid with 1 mL of the 0.5% methylcellulose media and the plates were incubated at 37° C./5% CO₂ for 3 days. Then the methylcellulose medium was removed and the wells were washed once with 1 mL PBS. After wash, the plates were fixed with ice cold methanol per well at −20° C. for 30 minutes. After fixation, the methanol was discarded, the monolayers stained with 0.2% crystal violet for 30 minutes at room temperature and then washed with PBS or dH₂O. Plates were let dry and the neutralizing antibody titer was identified as the highest serum dilution that reduced the number of virus plaques in the test by 50% or greater.

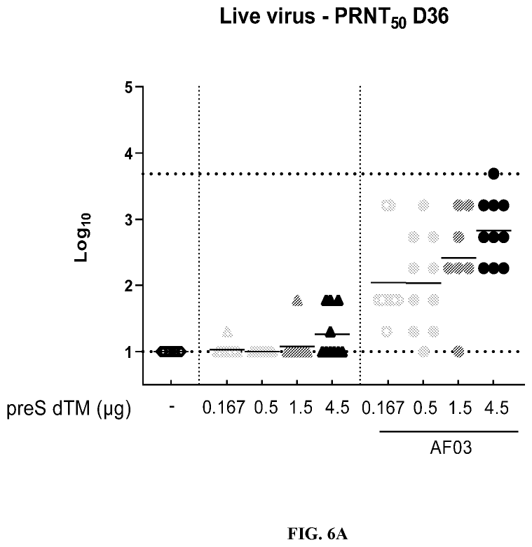

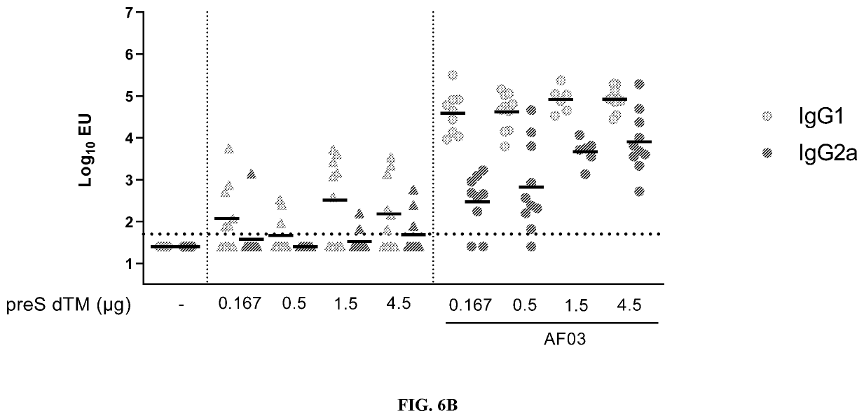

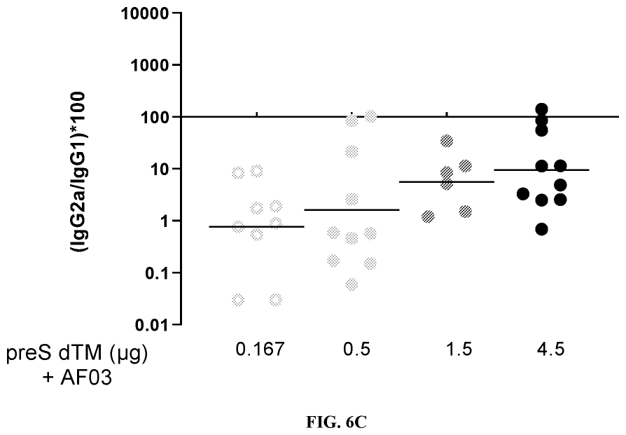

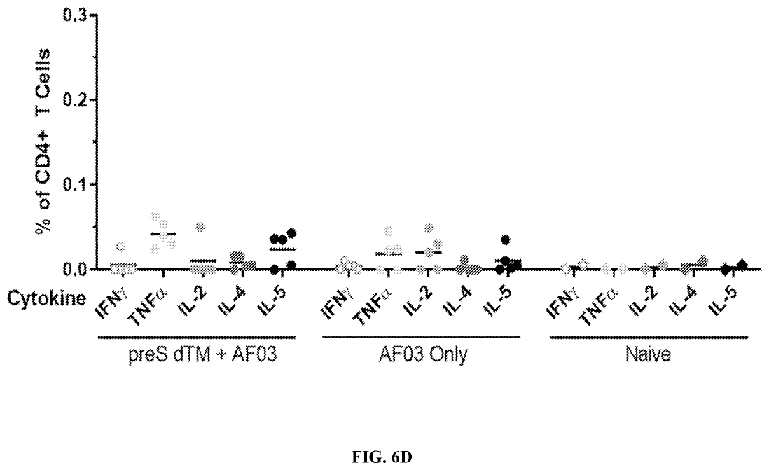

PRNT₅₀ titers were detected in all mice except one in the 0.5 μg group. Neutralizing mean range from 2.0 Log₁₀ in the lowest vaccine dose group (0.167 μg) to 2.9 Log₁₀ in the highest vaccine dose group (4.5 μg). Thus, animals immunized with the adjuvanted formulation generated a significantly higher amount of SARS-CoV-2 neutralizing antibodies by Day 36 in a dose-dependent manner than the non-adjuvanted groups (FIG. 6A).

The IgG₁ (associated with Th2) and IgG₂ₐ (associated to Th1) titers were measured on D36 in order to document the Th1/Th2 polarization profile responses. While the non-adjuvanted preS dTM vaccine elicited either no or very low IgG₁ and IgG₂ₐ responses, the AF03 adjuvanted preS dTM vaccine elicited robust IgG₁ responses in all vaccine doses (IgG₁ mean titers from 4.6 to 4.9 Log₁₀ EU). IgG₂ₐ were elicited at a lower level, and titers increased with the vaccine doses (mean titers from 2.5 to 3.9 Log₁₀ EU) (FIG. 6B). IgG₂a/IgG₁ ratio were calculated as an indication of the Th1/Th2 profile and showed a significantly higher ratio with increasing vaccine doses (p<0.05) (FIG. 6C).

### Example 4: Adjunct Mouse Study

This example describes a second mouse study of a SARS-CoV-2 recombinant protein vaccine formulation in mice. This study focused on evaluating cell-mediated immunity (CMI) in the immunized mice. The mice used here were female inbred BALB/c mice, 6-8 weeks old. They were injected intramuscularly with 50 μL of the vaccine formulation on Day 0 and Day 14. The dosing regimens are shown as follows, with five mice per group. The preS dTM injected was targeted at 4.5 μg, with or without adjuvant (AF03). For consistency, only the targeted doses are indicated in the text and figures.

TABLE 3
CoV2-03_Ms Targeted and Actual
Doses of CoV2 preS dTM Antigen

| Injection | Targeted | CoV2 preS dTM | Actual |
|---|---|---|---|
| day | doses (μg) | content %* | doses (μg) |
| D 0 | 4.5 | 41 | 1.8 |
| D 21 | 4.5 | 26 | 1.17 |

*Actual doses were re-calculated based on new assays which differentiate structurally correct CoV2 preS dTM trimers from HCP impurities. The assays are based on ACE2 binding and/or HPSEC.

Blood was drawn from the animals on Day 0, Day 14, and Day 24. Spleen was harvested on Day 24 for CMI analysis, and spleen cells were stimulated with S1+S2 15-mer peptide pools (JPT) having 11 amino acid overlap. Cells were phenotyped by flow cytometry approach and cytokine production was assessed by intracellular cytokine staining (ICS). The biomarker panel evaluated is shown below.

TABLE 4
CMI Biomarker Panel

| Antibody | Format | Clone | Vendor |
|---|---|---|---|
| CD3 | BUV395 | 17A2 | BD |
| CD4 | PerCP-Cy5.5 | RM4-5 | Biolegend |
| CD8 | AF700 | 53-6.7 | BD |
| IFN-γ | FITC | XMG1.2 | BD |
| IL-5 | PE | TRFK5 | Biolegend |
| TNF | Pacific Blue | MP6-XT22 | Biolegend |
| IL-4 | APC | 11B11 | Biolegend |
| CD45RA | PE-Cy7 | RA3-6B2 | BD |
| CD14 | PE-Cy7 | Sa14-2 | Biolegend |
| IL-2 | BV605 | JES6-SH4 | BD |
| LIVE/DEAD | Near-IR (APC-Cy7) | | ThermoFisher |

To perform intracellular staining (ICS), the spleens were homogenized, red blood cells were lysed and the cells were rested for 1 hour at 37° C. and 5% CO₂. The splenocytes were then counted and 2×10⁶ cells were incubated for 6 hours at 37° C. and 5% CO₂ with Golgi Plug (BD Biosciences) under four conditions: no peptide stimulation (media only control), positive control stimulation, and stimulation with two individual spike peptide pools (JPT product PM-WCPV-S-1). Cells from each individual animal were stimulated with a Cell Activation Cocktail with Brefeldin A (Biolegend) as a positive control. Following stimulation, cells were washed and resuspended in Mouse BD Fc Block™ (clone 2.4G2) for 10 minutes at 4° C. Cells were then spun down, Fc block was removed, and cells were surface stained and live/dead stained for 30 minutes at 4° C. with an antibody cocktail containing: CD4 (RM4-5) PerCP-Cy5.5 (Biolegend), CD8 (53-6.7) AF700 (BD Biosciences), CD45R/B220 (RA3-6B2) PE/Cy7 (BD Biosciences), CD14 (Sa14-2) PE/Cy7 (Biolegend) and LIVE/DEAD Fixable Near-IR Dead Cell Stain Kit (Invitrogen) in stain buffer (FBS) (BD Biosciences). After surface staining, cells were washed, fixed and permeabilized with Cytofix/Cytoperm solution (BD Biosciences) for 30 minutes at 4° C. Cells were then washed with 1×Perm/Wash solution (BD Biosciences), followed by intracellular staining for 30 minutes at 4° C., light protected, with a cocktail containing: CD3e (17A2) BUV395 (BD Biosciences), IFN-γ (XMG1.2) FITC (BD Biosciences), TNF-α (MP6-XT22) Pacific Blue (Biolegend), IL-2 (JES6-5H4) BV605 (BD Biosciences), IL-4 (11B11) APC (Biolegend), and IL-5 (TRFK5) PE (Biolegend) in 1×Perm/Wash buffer. Cells were then washed and resuspended in FACS buffer. Samples were run on an LSR Fortessa flow cytometer (BD Biosciences) and analysis was conducted on FlowJo software (version 10.6.1).

The ICS analysis indicated no or low frequency of S-specific CD4⁺ T cells expressing IFN-γ, TNF-α, IL-2, IL-4 and IL-5 in response to splenocyte stimulation with both S1 and S2 peptide pools in the AF03-adjuvanted vaccine immunized mice (below 0.5%, in the range of the non-specific signal detected in adjuvant-alone immunized mice (FIG. 6D). Response to S1 and S2 peptide pools was similar. Only response to S1 peptides is shown. No S-specific CD8⁺ T cell responses were detected (data not shown).

### Example 5: Non-Human Primate Study

This example describes a study in non-human primates (NHP) evaluating the humoral immunity. The animals used here were Rhesus macaques, 4-12 years old. The NHPs were injected with a targeted dose of 5 or 15 μg of preS dTM mixed with AF03 intramuscularly on Day 0 and Day 21 in a volume of 0.5 mL. Serum was collected on D4, D21, D28, and D35. On Day 56, the immunized animals were challenged with SAR2-CoV-2 USA/WA1/2020 strain at 10⁶ PFU through the intranasal (total of 1 mL) and intratracheal (total of 1 mL) routes. For consistency, only the targeted doses are indicated in the text and FIGURES.

TABLE 5
Dosing Regimen for NHP Study

| Group | Targeted Antigen | Adjuvant |
|---|---|---|
| (n = 6) | Dose (μg) | Dose (μg) |
| 1 | Diluent | — |
| 2 | preS dTM (15) | — |
| 3 | preS dTM (15) | AF03 (12,500) |
| 4 | preS dTM (5) | AF03 (12,500) |

TABLE 6
CoV2-02_NHP Targeted and Actual
Doses of CoV2 preS dTM Antigen

| Injection | Targeted | CoV2 preS dTM | Actual |
|---|---|---|---|
| day | doses (μg) | content %* | doses (μg) |
| D 0 | Low: 5 | 26 | Low: 1.3 |
| | High: 15 | | High: 3.9 |
| D 21 | Low: 5 | 14 | Low: 0.7 |
| | High: 15 | | High: 2.1 |

*Actual doses were re-calculated based on ACE2 binding quantification.

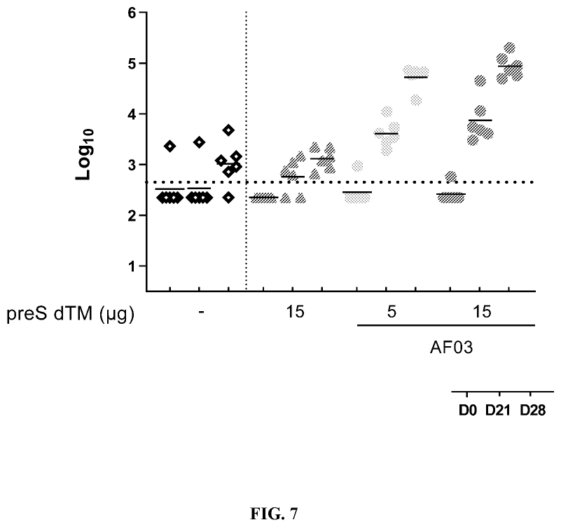

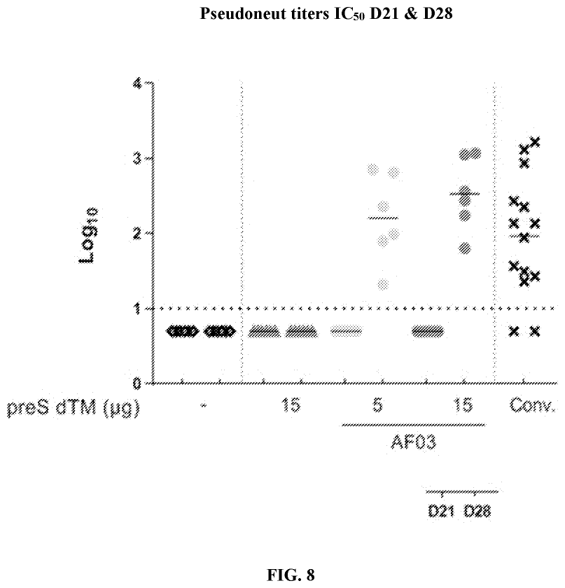

Consistent with the antibody responses observed in mice with preS dTM, no or very low responses were detected in absence of the adjuvant. However, when formulated in AF03 adjuvant, the vaccine elicited high levels of IgGs binding to the prefusion S in all immunized monkeys, as early as 2 weeks post-dose 1 (mean titers of 3.6 and 3.9 Log₁₀ EU in the 5 and 15 μg doses respectively). The second immunization potently increased the IgG titers on D28 (mean titers of 4.7 and 4.9 Log₁₀ EU in the 5 and 15 μg doses respectively). Importantly, the titers were not different among the two antigen dose groups (5 and 15 μg) (FIG. 7). The functional antibody responses elicited by the preS dTM vaccine was assessed using a GFP-pseudovirus (Integral Molecular) neutralization assay. Three weeks post-dose 1, no pseudovirus neutralizing titers were detected. However, one week after the second injection (D28), pseudovirus neutralizing titers were measured in all AF03-adjuvanted preS dTM immunized macaques (mean titers of 2.1 and 2.5 Log₁₀ IC₅₀ in the 5 and 15 μg groups respectively). No statistically significant differences between the two doses (5 and 15 μg) were evidenced. The functional antibody responses of the immunized rhesus were compared to the titers obtained from a panel of human convalescent sera (Conv.) and showed similar titers in the AF03-adjuvanted vaccine groups) (FIG. 8).

### Example 6: In Vitro Study in Primary Human Cells

This example describes a study that investigated the Th profiles induced by preS dTM with or without the AF03 adjuvant. In this study, pooled PBMCs from 50 human donors were primed with a targeted 2.5 or 5 μg dose of preS dTM with or without AF03 or another adjuvant. The adjuvant was provided in 250 μg/mL. The cells were then fixed and permeabilized, and stained with antibodies to cell surface markers and to cytokines characteristic of Th1 (IFN-γ, TNF-α, and IL-2) or Th2 (IL-4, IL-5 and IL-17) responses.

TABLE 7
In Vitro MIMIC Study Design

| Group | Targeted Antigen | Actual Antigen | |
|---|---|---|---|
| (n = 50) | doses (μg) | doses (μg) | Adjuvant |
| 1 | 0 | 0 | / |
| 2 | 2.5 | 1 | / |
| 3 | 5 | 2 | / |
| 4 | 2.5 | 1 | AF03 |
| 5 | 5 | 2 | AF03 |

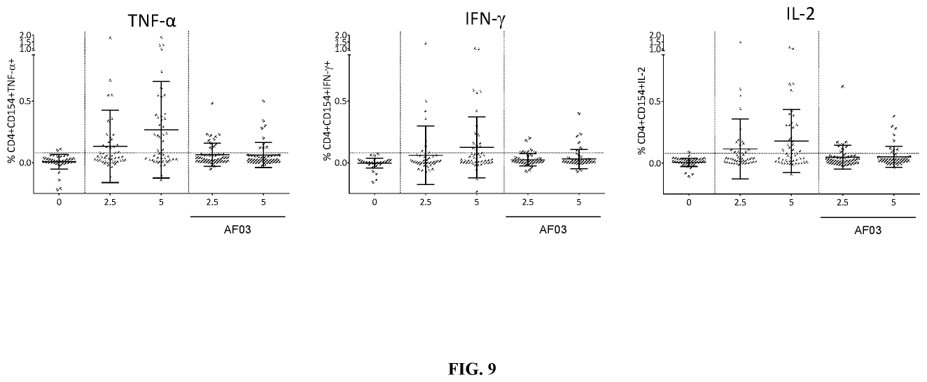

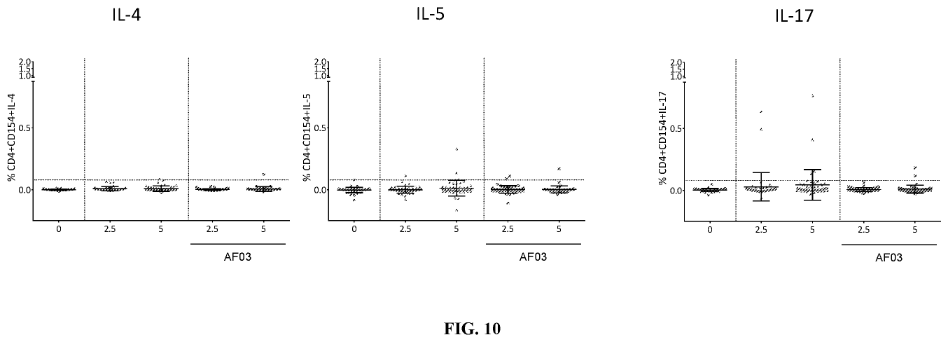

The data show that preS dTM induced a primarily Th1 response in the LTE, and no adjuvant effect was observed (FIG. 9 and FIG. 10).

### Example 7: Clinical Study

This example describes a Phase I/II clinical protocol for evaluating the safety and efficacy of a vaccine composition of the present disclosure. Participant, outcome assessors, investigators, laboratory personnel, and the majority of Sponsor study staff (except those involved in the ESDR and for concerned participants only) will be blinded to vaccine group assignment group (formulation and adjuvant; injection schedule will be unblinded). Those preparing/administering the study interventions will be unblinded to vaccine group assignment. Participants are randomized and stratified by age.

The composition comprises preS dTM (a trimer of a polypeptide of SEQ ID NO:10, without the signal peptide) with or without adjuvant. The vaccine composition is provided at two dosage strengths: Formulations 1 and 2, containing the CoV2 preS dTM antigen at 5 μg (low dose) and 15 μg (high dose), respectively. The antigen composition is shown below:

TABLE 8
Antigen Compositions for Clinical Study

| | Quantity | |
|---|---|---|
| Component | (per 0.25 mL Dose) | |
| Purified preS dTM recombinant | 5 or 15 | μg |
| protein | | |
| Sodium chloride | 4.4 | mg |
| Monobasic sodium phosphate | 0.195 | mg |
| Dibasic sodium phosphate | 1.3 | mg |
| Polysorbate 20 (Tween ® 20) | 27.5 | μg |
| Baculovirus and Spodoptera | ≤19 | μg |
| frugiperda cell proteins | | |
| Baculovirus and cellular DNA | ≤10 | ng |
| Triton X-100 | ≤100 | μg |

For a subsequent study, the antigen may be provided in an aqueous liquid solution, prior to mixture with any adjuvant, as shown in Table 8A below.

TABLE 8A
Antigen Formulation

| Role of drug | Name of drug | Quantity | | |
|---|---|---|---|---|
| ingredient/substance | ingredient/substance | expression | Quantity per dose | |
| Antigen | Recombinant preS dTM protein | ≥(not | 2-50 (e.g., 2.5, | |
| | (SARS-CoV-2 Wuhan D614 strain) | less than) | 5, 10 or 15) μg | |
| Excipient - Buffer | Monobasic sodium phosphate | Equal | 0.0975 | mg |
| Excipient - Buffer | Dibasic sodium phosphate | Equal | 0.65 | mg* |
| | dodecahydrate | | | |
| Excipient - Buffer | Sodium chloride | Equal | 2.2 | mg |
| Excipient - Buffer | Polysorbate 20 | Equal | 55 | μg |
| Solvent/Diluent | Water for injection | Sufficient | 0.25 | mL |
| | | quantity (q.s.) | | |
| Excipient - Adjuvant | If used | Sufficient | 0.25 | mL |
| | | quantity | | |

*This corresponds to 0.26 mg dibasic sodium phosphate anhydrous, which can also be used to prepare the formulation.

To evaluate the effect of adjuvant, AF03 is used. The unit dose strength for the adjuvant study groups is 5 μg and 15 μg, of preS dTM. Each mono-dose vial of the squalene-based AF03 contains ingredients shown below.

TABLE 9
Adjuvant Composition for Clinical Study

| | |
|---|---|
| Adjuvant | AF03 Quantity (per 0.25 mL Dose) |

| | | |
|---|---|---|
| Oily phase | Squalene | 12.5 mg |
| Lipophilic surfactant | Sorbitan monooleate | 1.85 mg |
| | (Dehymuls SMO ™) | |
| Hydrophilic surfactant | POE (12) | 2.38 mg |
| | Cetostearyl ether | |
| | (Kolliphor CS12 ™) | |
| Additional excipient | Mannitol | 2.31 mg |
| Additional Buffer | PBS Buffered aqueous phase | pH 7.2 |

| | |
|---|---|
| Average particle size | 100 nm |
| Manufacturing process | Phase Inversion Temperature |

The antigen composition and the adjuvant composition are mixed prior to use, with a total volume of 0.5 mL. Placebo is 0.5 mL per dose of 0.9% normal saline.

The route of administration is intramuscular injection, at the deltoid muscle in the upper arm.

Each study intervention will be provided in an individual box (antigen and adjuvant or antigen and diluent (PBS) will be kitted together in a 2-vial box).

Participants are 18 years of age and older, healthy individuals and randomized within age groups. A small sentinel cohort made up of participants 18-49 years of age (Cohort 1) will receive a single dose. If safety data and laboratory measures to D09 in Cohort 1 are considered as acceptable based on unblinded data review, the remaining participants in Cohort 1 and all participants in Cohort 2 will be enrolled. All participants will receive one injection of either one of the investigational study vaccine formulations or the placebo control at D01 (Vaccination [VAC] 1). Participants in Cohort 2 will receive a second injection of study vaccine formulation or placebo at D22 (VAC2). The duration of each participant's participation in the study will be approximately 365 days post-last injection.

COVID-19-like illness will be part of efficacy objective with active and passive surveillance. It is anticipated that the design of the candidate SARS-CoV-2 antigen selected for this study will promote generation of robust neutralizing antibodies over binding antibodies. The inclusion of adjuvanted formulations is anticipated to further enhance the magnitude of neutralizing antibody responses and induce a balanced Th1/Th-2 T-helper cell responses. Taken together, these strategies mitigate by design theoretical risks of immune enhancement of viral infection. Individuals with chronic comorbid conditions considered to be associated with an increased risk of severe COVID-19 will be excluded.

A primary objective of the study is to evaluate immunogenicity of the vaccine composition by describing the levels and profiles of neutralizing antibodies at D01, D22, and D36. Neutralizing antibody titers will be measured with the neutralization assay. It is expected that the serum antibody neutralization titer post-vaccination at D22 and D36 will increase by about 2- to 4-fold relative to D01. Occurrence of neutralizing antibody seroconversion is defined as values below lower limit of quantification (LLOQ) at baseline with detectable neutralization titer above assay LLOQ at D22 and D36.

A secondary objective of the study is to evaluate immunogenicity of the vaccine composition by describing the binding antibody profile at D01, D22, D36, D181 (Cohort 1) or D202 (Cohort 2), and D366 (Cohort 1) or D387 (Cohort 2) of each study intervention group, and describing the neutralizing antibody profile at D181 (Cohort 1) or D202 (Cohort 2) and D366 (Cohort 1) or D387 (Cohort 2) of each study intervention group. Binding antibody titers to full-length SARS-CoV-2 spike protein will be measured for each study intervention group with the enzyme-linked immunosorbent assay (ELISA) method. It is expected that the fold-rise in anti-S antibody concentration [post/pre] will be 2 or more, or 4 or more at D22, D36, D181 (Cohort 1) or D202 (Cohort 2), and D366 (Cohort 1) or D387 (Cohort 2). Neutralizing antibody titers will be measured with the neutralization assay. It is expected that fold-rise in serum neutralization titer postvaccination at D181 (Cohort 1) or D202 (Cohort 2) and D366 (Cohort 1) or D387 (Cohort 2) relative to D01 will be 2 or more or 4 or more. Occurrence of neutralizing antibody seroconversion is defined as values below LLOQ at baseline with detectable neutralization titer above assay lower limit of quantification at D181 (Cohort 1) or D202 (Cohort 2) and D366 (Cohort 1) or D387 (Cohort 2).

Another secondary object of the study is to evaluate efficacy by describing the occurrence of virologically-confirmed COVID-19-like illness and serologically confirmed SARS-CoV-2 infection and evaluating the correlation/association between antibody responses to SARS-CoV-2 Recombinant Protein and the risk of COVID-19-like illness and/or serologically confirmed SARS-CoV-2 infection. Virologically confirmed COVID-19-like illness is defined by specified clinical symptoms and signs and confirmed by nucleic assay viral detection assay. Serologically-confirmed SARS-CoV-2 infection is defined by SARS-CoV-2-specific antibody detection in a non-S ELISA. Risk/protection correlation is based on antibody responses to SARS-CoV-2 as evaluated using virus neutralization or ELISA, considering virologically confirmed COVID-19 like illness and/or serologically confirmed SARS-CoV-2 infection as defined above.

An exploratory objective of the study is to evaluating immunogenicity by describing cellular immune response profile at D22 and D36 for each study intervention group in Cohort 2 and describing the ratio between neutralizing antibodies and binding antibodies. Th1 and Th2 cytokines will be measured in whole blood and/or cryopreserved PBMC following stimulation with full-length S protein and/or pools of S-antigen peptides. Ratio between binding antibody (ELISA) concentration and neutralizing antibody titer will be calculated.

SARS-CoV-2 Neutralizing Antibody Assessment

SARS-CoV-2 neutralizing antibodies will be measured using a neutralization assay. In this assay, serum samples are mixed with constant concentration of the SARS-CoV-2 virus. A reduction in virus infectivity (viral antigen production) due to neutralization by antibody present in serum samples can be detected by ELISA. After washing and fixation, SARS-CoV-2 antigen production in cells can be detected by successive incubations with an anti-SARS-CoV-2-specific antibody, HRP IgG conjugate, and a chromogenic substrate. The resulting optical density is measured using a microplate reader. The reduction in SARS-CoV-2 infectivity as compared to that in the virus control wells constitutes a positive neutralization reaction indicating the presence of neutralizing antibodies in the serum sample.

SARS-CoV-2 Spike Protein Antibody Serum IgG ELISA

SARS-CoV-2 anti-S protein IgG antibodies will be measured using an ELISA. Microtiter plates will be coated with SARS-CoV-2 spike protein antigen diluted in coating buffer to the optimal concentration. Plates may be blocked by the addition of a blocking buffer to all wells and incubation for a defined period. Following incubation, plates will be washed. All controls, reference, and samples will be pre-diluted with dilution buffer. The pre-diluted controls, reference and samples will then be further serially diluted in the wells of the coated test plate. The plates will be incubated for a defined period. Following incubation, plates will be washed, an optimized dilution of goat anti-human IgG enzyme conjugate will be added to all wells, and plates will be further incubated. Following this incubation, the plates will be washed, and enzyme substrate solution will be added to all wells. Plates will be incubated for a defined period to allow the substrate to develop. Substrate development will be stopped by the addition of a stop solution to each well. An ELISA microtiter plate reader will be used to read the test plates using assay specific SoftMax Pro templates. The average optical density (OD) value for the plate blank will be subtracted from all the ODs within each plate. The sample titers will be derived using the measured values of the blanks, controls, and the reference standard curve, which will be included on each assay plate within the run.

Cellular-Mediated Immunity (Using Whole Blood and/or PBMCs)

Cytokines will be measured in whole blood and/or cryopreserved PBMCs following stimulation with full-length S protein and/or pools of S-antigen peptides.

COVID-19-Like Illness

COVID-19-like illness is defined as having (i) any one of the following (that persist for a period of at least 12 hours or reoccur within a 12-hour period): cough (dry or productive); anosmia; ageusia; chilblains (COVID-toes); difficulty breathing or shortness of breath; clinical or radiographic evidence of pneumonia; and any hospitalization with the clinical diagnosis of stroke, myocarditis, myocardial infarction, thromboembolic events (e.g., pulmonary embolism, deep vein thrombosis, and stroke), and/or purpura fulminans; or (ii) any two of the following (that persist for a period of at least 12 hours or reoccur within a 12-hour period): pharyngitis; chills; myalgia; headache; rhinorrhea; abdominal pain; and at least one of nausea, diarrhea, and vomiting.

Virologically Confirmed COVID-19 Illness

Virologically confirmed COVID-19 illness is defined as a positive result for SARS-CoV-2 by Nucleic Acid Amplification Test (NAAT) on a respiratory sample in association with a COVID-19-like illness.

Serologically Confirmed SARS-CoV-2 Infection

Serologically confirmed SARS-CoV02 infection is defined as a positive result in serum for presence of antibodies specific to non-Spike protein of SARS-CoV-2 detected by ELISA.

SARS-CoV-2 Nucleoprotein Antibody Serum IgG ELISA

SARS-CoV-2 anti-nucleoprotein antibodies will be measured using an ELISA. Microtiter plates will be coated with SARS-CoV-2 nucleoprotein antigen diluted in coating buffer to the optimal concentration. Plates may be blocked by the addition of a blocking buffer to all wells and incubation for a defined period. Following incubation, plates will be washed. All controls, reference, and samples will be pre-diluted with dilution buffer. The pre-diluted controls, reference and samples will then be further serially diluted in the wells of the coated test plate. The plates will be incubated for a defined period. Following incubation, plates will be washed, an optimized dilution of goat anti-human IgG enzyme conjugate will be added to all wells, and plates will be further incubated. Following this incubation, the plates will be washed, and enzyme substrate solution will be added to all wells. Plates will be incubated for a defined period to allow the substrate to develop. Substrate development will be stopped by the addition of a stop solution to each well. An ELISA microtiter plate reader will be used to read the test plates using assay specific SoftMax Pro templates. The average OD value for the plate blank will be subtracted from all the ODs within each plate. The sample titers will be derived using the measured values of the blanks, controls, and the reference standard curve, which will be included on each assay plate within the run.

Nucleic Acid Amplification Test (NAAT) for COVID-19 Case Detection

In the assay, respiratory samples will be collected and the RNA is extracted. The purified template is then evaluated by an NAAT using SARS-CoV-2 specific primers to specifically amplify SARS-CoV-2 targets.

### Example 8: Use of the Recombinant S Vaccine as Part of a Prime-Boost Regimen

Recent studies have shown that humoral responses against SARS-CoV-2 build up rapidly, peaking at about week 2 or 3 after symptoms onset, but decline steadily in the next three months (see, e.g., Beaudoin-Bussieres et al., mBio (2020) 11(5):e02590-20; Altmann and Boyton, Sci Immunol. (2020) 5(49):eabd6160; Hellerstein, Vaccine X (2020) 6:100076j; Seow et al., Nat Microbiol. (2020) 5:1598-1607; Tan et al., Front Med. (2020) 5:1-6). These findings suggest that the early dynamics of humoral response to SARS-CoV-2 is similar to those for other acute viral infections. It has been reported that the binding antibody concentrations and SARS-CoV-2 neutralizing titers elicited by two doses of mRNA vaccine also follow this pattern, showing a decline in five weeks after the second dose (Sahin et al., Nature (2020) 586:594-9; Mulligan et al., Nature (2020) 586:589-593).

This Example describes a study in which a protein vaccine of the present disclosure was used as a booster for an mRNA vaccine, mRNA-VAC1 or mRNA-VAC2, in an NHP model. mRNA-VAC1 and mRNA-VAC2 are mRNA vaccines. They both encode a recombinant S protein whose polypeptide sequence is SEQ ID NO:13 but contain different lipid nanoparticle formulations. mRNA-VAC1 has been shown to induce binding and neutralizing antibodies, as well as Th1-biased T cell responses in mice and NHPs (biorxiv.org/content/10.1101/2020.10.14.337535v1).

Materials and Methods

Enzyme-Linked Immunosorbent Assay (ELISA)

Nunc MaxiSorb plates were coated with SARS-CoV S-GCN4 protein (custom made at GeneArt) protein at 0.5 μg/mL in PBS overnight at 4° C. Plates were washed 3 times with PBS-Tween 0.1% before blocking with 1% BSA in PBS-Tween 0.1% for 1 hour at ambient temperature. Samples were plated with 1:450 initial dilution followed by 3-fold, 7-point serial dilution in blocking buffer. Plates were washed 3 times after 1-h incubation at room temperature before adding 50 μL of 1:5000 Rabbit anti-human IgG (Jackson Immuno Research) to each well. Plates were incubated at room temperature for 1 hour and washed 3×. Plates were developed using Pierce 1-Step™ Ultra TMB-ELISA Substrate Solution for 0.1 hour and stopped by TMB stop solution. Plates were read at 450 nm in SpectraMax® plate reader. Antibody titers were reported as the highest dilution that is >0.2 Optical Density (OD) cutoff.

Pseudovirus Neutralization Assay

Serum samples were diluted 1:4 in media (FluoroBrite™ phenol red free DMEM+10% FBS+10 mM HEPES+1% PS+1% GlutaMAX™) and heat-inactivated at 56° C. for 0.5 hour. A further, 2-fold serial dilution of the heat-inactivated serum were prepared and mixed with the reporter virus particle (RVP)-GFP (Integral Molecular) diluted to contain 300 infectious particles per well and incubated for 1 hour at 37° C. 96-well plates of 50% confluent 293T-hsACE2 clonal cells in 75 μL were inoculated with 50 μL of the serum/virus mixtures and incubated at 37° C. for 72 hours. At the end of the incubation, plates were scanned on a high-content imager and individual GFP expressing cells were counted. The inhibitory dilution titer (ID₅₀) was reported as the reciprocal of the dilution that reduced the number of virus plaques in the test by 50%. ID50 for each test sample was interpolated by calculating the slope and intercept using the last dilution with a plaque number below the 50% neutralization point and the first dilution with a plaque number above the 50% neutralization point. ID50 Titer=(50% neutralization point−intercept)/slope).

Microneutralization Assay

Serial two-fold dilutions of heat-inactivated serum samples were incubated with a challenge dose targeting 50% tissue culture infectious dose (TCID₅₀) of SARS-CoV-2 (strain USA-WA1/2020 [BEI Resources; catalog #NR-52281]) at 37° C. with 5% CO₂ for 1 hour. The serum-virus mixtures were inoculated into wells of a 96-well microplate with preformed Vero E6 (ATCC® CRL-1586™) cell monolayers and adsorbed at 37° C. with 5% CO₂ for 0.5 hour. Additional assay medium was added to all wells without removing the existing inoculum and incubated at 37° C. with 5% CO₂ for 2 days. After washing and fixation of the Vero E6 cell monolayers, SARS-CoV-2 antigen production in cells was detected by successive incubations with an anti-SARS-CoV nucleoprotein mouse monoclonal antibody (Sino Biological, catalog #40143-MM05), HRP IgG conjugate (Jackson ImmunoResearch Laboratories, catalog #115-035-062), and a chromogenic substrate. The resulting optical density (OD) was measured using a microplate reader. The reduction in SARS-CoV-2 infectivity, as compared to that in the virus control wells, constitutes a positive neutralization reaction indicating the presence of neutralizing antibodies in the serum sample. The 50% neutralization titer (MN ID₅₀) was defined as the reciprocal of the serum dilution for which the virus infectivity was reduced by 50% relative to the virus control on each plate. The MN ID₅₀ for each sample was interpolated by calculating the slope and intercept using the last dilution with an OD below the 50% neutralization point and the first dilution with an OD above the 50% neutralization point; MN ID₅₀ Titer=(OD of 50% neutralization point-intercept)/slope.

Memory B Cell Analysis

For testing B cell memory responses in NHPs, monkey IgG/IgA FluoroSpot Kit (Kit; MABTECH, Cat #FS-05R24G-10) was used. Frozen PBMCs were washed in the culture medium (RPMI 1640 with L-glutamine, 10% FCS and 1% penicillin-streptomycin), re-suspended in a petri dish in the same culture medium supplemented with R848 and recombinant human IL-2 (rhIL-2) at final concentrations of 1 μg/mL and 10 ng/mL, respectively, and incubated at 37° C. with 5% CO₂ for 3 days. The FluoroSpot plates were coated overnight with 4 μg/mL of SARS-CoV2 S-GCN4 protein (GeneArt) or 15 μg/mL of anti-IgG and IgA mAbs provided in the Kit. The plates were then blocked by the complete medium for 1 hour. Pre-stimulated PBMCs were washed, counted to determine the number of viable cells and added to the plates at 5×10⁵ cells per well for the wells coated with SARS-CoV2 S-GCN4 protein and at 1×10⁵ cells per well for the wells coated with anti-IgG and IgA mAbs. The plates were incubated at 37° C. with 5% CO₂ for 16-24 hours. After washing, fluorescently labeled anti-IgG and IgA detection antibodies were added for 2 hours followed by the fluorescence enhancer. The fluorescent spots were counted with a FluoroSpot analyzer after the plate was air-dried for 24 hours. The data were reported as a number of antibody-secreting cells (ASC) per million PBMCs.

Cytokine ELISPOT Analysis

For testing cytokine responses in NHPs, monkey IFN-γ ELISPOT (CTL, cat #3421M-4APW) and IL-13 ELISPOT kits (CTL, cat #3470M-4APW) were used. Previously frozen PBMCs were washed, resuspended in culture medium provided by the kit and enumerated. PepMix™ SARS-CoV-2 peptide pools as well as CovA were used for stimulation. PBMC were plated at 300,000 cells per well and stimulated overnight. After overnight incubation, the plates were washed and developed per manufacturer instructions. The plates were dried overnight, scanned, and spots were counted using a CTL analyzer (ImmunoSpot® S6 Universal Analyzer, CTL). The data were reported as spot forming cells (SFC) per million PBMCs.

### Results

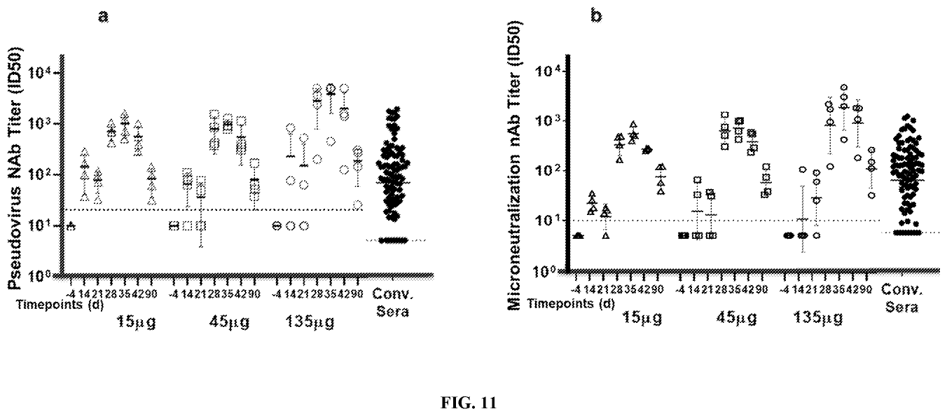

In the present study, cynomolgus macaques of Mauritian origin 2-6 years of age and weighing in a range of 2-6 kg were injected intramuscularly into the deltoid of the right forelimb with mRNA-VAC2 (15 μg, 45 μg, or 135 μg) on day 0 (DO) and then the other forelimb for the same amount of mRNA-VAC2 on day 21 (D21). NHP sera samples were collected on D4, 14, 21, 28, 35, 42 and, peripheral blood mononuclear cells (PBMCs) were isolated on D42. The data reveal a dramatic decline of neutralizing activities in sera samples on D90 (FIG. 11). Convalescent human sera obtained from commercial vendors (Sanguine Biobank, iSpecimen, and PPD) were included in assessment of immune responses in all assays. The antibody titers dropped to the levels corresponding to the single dose immunizations.

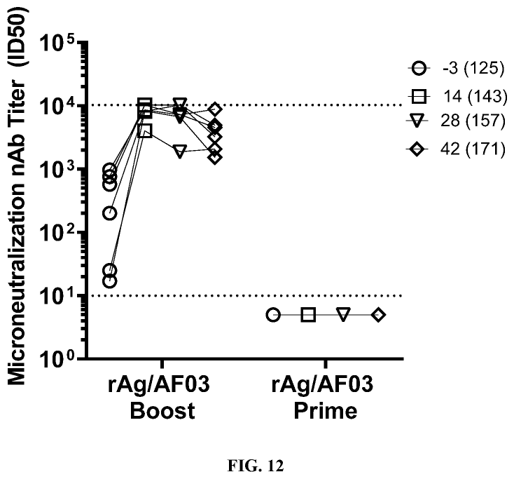

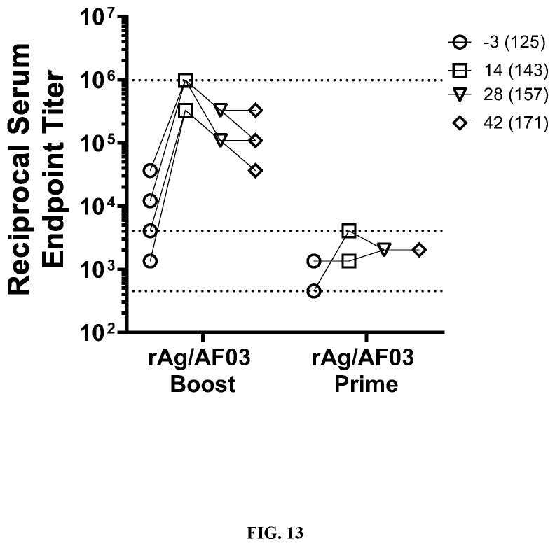

On D129, six mRNA-VAC2-immunized animals were boosted with preS dTM (3 μg) adjuvanted with AF03. On D14 post-boost (D143), the booster induced a robust 10-fold to 15-fold increase in microneutralization (MN₅₀) (FIG. 12) titers and binding antibody titers (FIG. 13). A non-significant decline of MN and binding titers from 2 to 6 weeks post-boost was observed.

We also explored whether primary responses provided B-cell memory component. We performed ELISPOT analysis of NHP PBMC samples collected before boost administrations (D90). Spike-specific memory B-cells in PBMCs from individual NHPs were enumerated on D90 following immunization with mRNA-VAC1 on DO (Table 10). Total and SARS-CoV-2 S-specific IgG ASC were enumerated by ELISPOT as described above. IgG specific activity was calculated as (S-specific IgG ASC/total IgG memory ASC)×100%.

TABLE 10
Spike-Specific Memory B Cells in PBMCs
from Individual NHPs on D 90

| | mRNA-VAC1 | % of S-Specific |
|---|---|---|
| NHP ID | Prime Dose (μg) | IgG ASC |
| 1 | 15 | 1.5 |
| 2 | 15 | 0.6 |
| 3 | 15 | 0.8 |
| 4 | 15 | 0.8 |
| 5 | 45 | 2.3 |
| 6 | 45 | 1.9 |
| 7 | 45 | 1.1 |
| 8 | 45 | 0.7 |
| 9 | 135 | 0.8 |
| 10 | 135 | 2.7 |
| 11 | 135 | 0.8 |
| 12 | 135 | 1.1 |

The data in Table 10 reveal that the level of circulated memory B-cells in mRNA-VAC1-immunized animals on D90 was from 0.6% to 4%, irrespective of the prime dosage used. This level was 5-10 fold higher than that reported for other vaccines (Scherer et al., PLoS Pathog. (2014) 10(12):e1004461; Weinberg et al., Hum Vaccin Immunother. (2019) 15:2466-74). This level of memory B cells is also consistent with recent reports on immunological memory assessment in COVID-19 patients six months after infection.

Table 11 shows the levels of spike-specific memory B Cells in PBMCs from individual NHPs before and after the boost with 3 μg of preS dTM adjuvanted with AF03. Prior to boost on D129, these NHPs were injected with mRNA-VAC2 on D0 and D21 at doses indicated in the table. Total and SARS-CoV-2 S-specific IgG ASC were enumerated by ELISPOT as described above. IgG specific activity was calculated as (S-specific IgG ASC/total IgG memory ASC)×100%.

TABLE 11
Spike-Specific Memory B Cells in NHP
PBMCs Before and After preS dTM Boost

| | |
|---|---|
| mRNA-VAC2 | % of S-Specific IgG ASC |

| | Prime Dose | D 90 (D 39 | D 171 (D 42 |
|---|---|---|---|
| NHP ID | (μg) | before boost) | after boost) |
| 13 | 135 | 2.6 | Not determined |
| 14 | 135 | 4.0 | 1.7 |
| 15 | 135 | 2.5 | 1.3 |
| 16 | 135 | 1.0 | 1.0 |
| 17 | 15 | 1.0 (0.5)* | 1.7 |
| 18 | 15 | 2.0 | 6.7 |

*Number in parenthesis: data from repeat experiment.

The data in Table 11 show that the memory B cell level induced by mRNA vaccination was not impacted in a dose-dependent manner by the prime dosage, despite visible increase in neutralization and binding antibody titers. The results in Table 11 suggest that the level of memory B cells elicited by mRNA vaccination was very high and might not be significantly boosted efficiently by a subunit vaccination. This finding could also be due to the short observation period for the experimentation (less than 6 months post vaccination).

Next, we investigated the T cell response profiles in the vaccinated NHPs. Vaccine-associated enhanced respiratory disease (VAERD) has been a safety concern for COVID-19 vaccines in development, although the concern at this stage is only a theoretical one (see, e.g., Graham et al., Science (2020) 368:945-6). VAERD has been reported for whole-inactivated virus vaccines against measles and respiratory syncytial virus (RSV) (Graham, supra). One explanation for VAERD implicates the biased production of Th2 cytokines (e.g., IL-4, IL-5, and IL-13) by antigen-specific CD4⁺ T cells. A similar association between a Th2 profile and disease enhancement has been reported for an inactivated SARS-CoV-1 vaccine in mice (Bolles et al., J Virol. (2011) 85:12201-5; Tseng et al., PLoS One (2012) 7:e35421). Less severe cases of SARS were associated with accelerated induction of Th1 cell responses (Oh et al., Emerg Microbes Infect. (2012) 1:1-6). Similar phenomena have been observed in humans. For example, a SARS-CoV-2-specific cellular response was associated with severity of disease: PBMCs from recovered patients with mild COVID-19 symptoms demonstrated high levels of IFN-γ induction by SARS-CoV-2 antigens, while PBMCs from COVID-19 patients with severe pneumonia showed significantly lower level of this cytokine (Kroemer et al., J Infect. (2020) 4816, doi:10.1016/j.jinf.2020.08.036). Thus, it is important to understand the T cell profiles induced by the present vaccination regimen.

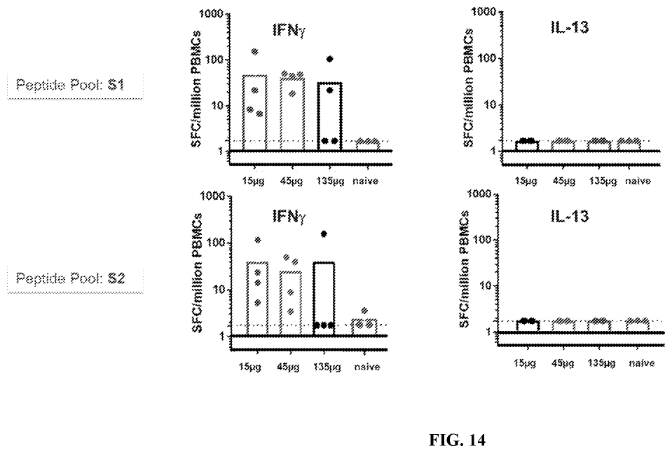

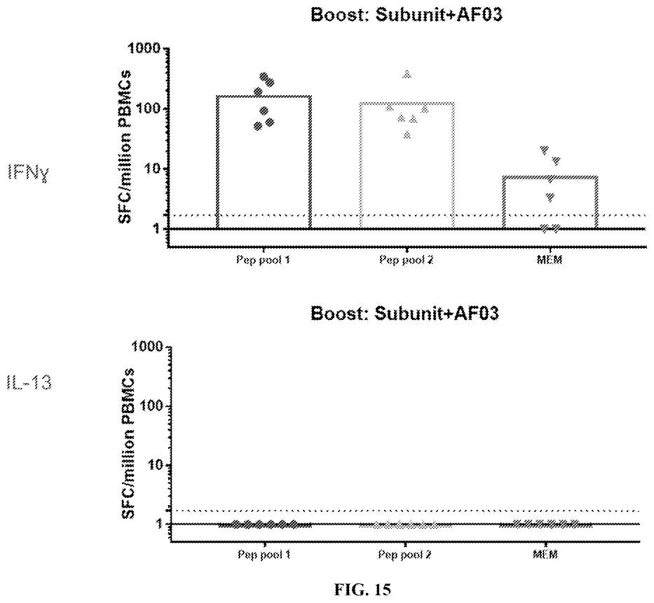

T cell cytokine responses were tested in NHPs three weeks after the second mRNA-VAC1 vaccination on D21. Cytokines induced by re-stimulation with the pooled SARS-CoV-2 S protein peptides were assessed in PBMCs on D42 by the IFN-γ (Th1 cytokine) and IL-13 (Th2 cytokine) ELISPOT assays. The majority of animals in three dose level groups tested (10 out of 12) demonstrated presence of IFN-γ secreting cells, ranging from two to over 100 spot-forming cells per million PBMCs. A dose-dependent response was not observed, as the animals in the lower and higher dose level groups showed comparable frequencies of IFN-γ secreting cells. In contrast, IL-13 cytokine secreting cells were not detected in any of the groups tested and at any dose level, suggesting induction of a Th1-biased cellular responses (FIG. 14). These data presented clear evidence for a lack of Th2 response to S antigen following mRNA-VAC1 vaccination in NHPs. We then examined the cytokine secretion profiles of PBMCs from animals boosted with preS dTM on D129. On D171 (D42 post-boost), the PMBCs from boosted animals maintained aTh1/Th2 ratio similar to the pre-boost ratio (FIG. 15).

These results demonstrate that the primary humoral memory response induced by two intramuscular of mRNA-VAC1 or a similar mRNA formulation (e.g., mRNA-VAC2) immunizations given 3 weeks apart was effectively boosted by a single administration on D129 with a single dose of adjuvanted protein vaccine formulations. In conclusion, the prime-boost regimen described herein allowed rapid reappearance of antibody in the blood following initial introduction of S-immunogen delivered via a genetic vaccine. These results suggest that the present protein vaccine can be introduced into the COVID-19 vaccine routine as a booster to provide durable and highly effective protection within pre-immune population.

### LIST OF SEQUENCES

| SEQ ID NO | Description |
|---|---|
| 1 | SARS-CoV-2 S protein amino acid sequence |
| | (Wuhan) |
| 2 | SARS-CoV-2 S protein signal peptide amino |
| | acid sequence |
| 3 | Mutant chitinase signal peptide amino acid |
| | sequence |
| 4 | SARS-CoV-2 S protein ectodomain amino acid |
| | sequence |
| 5 | RRAR |
| 6 | GSAS |
| 7 | Foldon amino acid sequence |
| 8 | Wildtype foldon coding sequence |
| 9 | Codon-optimized foldon coding sequence |
| 10 | preS dTM amino acid sequence |
| 11 | Wildtype chitinase signal peptide amino acid |
| | sequence |
| 12 | Polyhedrin promoter burst sequence |
| 13 | Exemplary recombinant SARS-CoV-2 S protein |
| 14 | Recombinant S protein amino acid sequence |
| | derived from B.1.351 |
| 15 | gBlock pPSC12-DB 3′ fragment sequence |
| 16 | F1-gBlock-5′ fragment sequence |
| 17 | F1-gBlock-3′ fragment sequence |
| 18 | F2-gBlock-5′ fragment sequence |
| 19 | F2-gBlock-3′ fragment sequence |
| 20 | F3-gBlock-5′ fragment sequence |
| 21 | F3-gBlock-3′ fragment sequence |
| 22 | gBlock pPSC12-DB 5′ fragment 1 sequence |
| 23 | F3b-gBlock-3′ fragment sequence |
| 24 | gBlock pPSC12-DB 5′ fragment 2 sequence |

1. An isolated polypeptide comprising, from N terminus to C terminus,
(i) a sequence that is at least 95% identical to residues 19-1243 of SEQ ID NO: 10, wherein residues GSAS (SEQ ID NO:6) at positions 687-690 of SEQ ID NO: 10 and residues PP at positions 991 and 992 of SEQ ID NO:10 are maintained in the sequence; and
(ii) a trimerization domain, wherein the trimerization domain comprises SEQ ID NO:7.

2. The isolated polypeptide of claim 1, further comprising (iii) a signal peptide derived from an insect or baculoviral protein, optionally wherein the insect or baculoviral protein is a chitinase.

3. The isolated polypeptide of claim 2, wherein the signal peptide comprises SEQ ID NO:3.

4. The isolated polypeptide of claim 1, wherein the polypeptide comprises or has a sequence identical to (i) residues 19-1243 of SEQ ID NO:10, or (ii) residues 19-1240 of SEQ ID NO:14.

5. A recombinant SARS-CoV-2 S protein, wherein the protein is a trimer of the polypeptide of claim 1.

6. The recombinant protein of claim 5, wherein the protein is a trimer of a polypeptide having the sequence identical to (i) residues 19-1243 of SEQ ID NO:10, or (ii) residues 19-1240 of SEQ ID NO:14.

7. A nucleic acid molecule encoding the polypeptide of claim 1, optionally wherein the nucleic acid molecule comprises SEQ ID NO:9.

8. A baculoviral vector for expressing a polypeptide, comprising the nucleic acid molecule of claim 7.

9. The baculoviral vector of claim 8, wherein expression of the polypeptide is under the control of a polyhedrin promoter.

10. A method of producing a recombinant SARS-CoV-2 S protein, comprising
introducing the baculoviral vector of claim 8 into insect cells,
culturing the insect cells under conditions that allow expression and trimerization of the polypeptide, and
isolating the recombinant SARS-CoV-2 S protein from the culture, wherein the recombinant SARS-CoV-2 S protein is a trimer of the polypeptide, without the signal sequence.

11. A recombinant SARS-CoV-2 S protein produced by the method of claim 10.

12. An immunogenic composition comprising one, two, three, or more recombinant SARS-CoV-2 S proteins of claim 5, and a pharmaceutically acceptable carrier, optionally wherein the pharmaceutically acceptable carrier comprises a phosphate-buffered saline comprising 7.5 mM phosphate and 150 mM NaCl, pH 7.2, and optionally 0.2% polysorbate 20 (Tween 20®).

13. An immunogenic composition comprising one, two, three, or more recombinant SARS-CoV-2 S proteins of claim 5, and for every 0.25 mL or each dose of the composition, the composition comprises or is prepared by mixing
a total of 2 μg to 50 μg of the recombinant SARS-CoV-2 S protein(s),
0.097 mg sodium phosphate monobasic monohydrate,
0.26 mg sodium phosphate dibasic anhydrous,
2.2 mg sodium chloride,
550 μg polysorbate 20, and
about 0.25 mL water.

14. The immunogenic composition of claim 12, wherein each dose of the composition comprises or is prepared by mixing:
(i) an antigen component comprising a total of about 2 to about 45 μg of the recombinant SARS-CoV-2 S protein(s); and
(ii) one dose of an adjuvant, wherein each dose of the adjuvant is 0.25 mL in volume and comprises or is prepared by mixing
12.5 mg squalene,
1.85 mg sorbitan monooleate,
2.38 mg polyoxyethylene cetostearyl ether,
2.31 mg mannitol, and
a phosphate-buffered saline.

15. The immunogenic composition of claim 12, comprising a total of 2.5, 5, 10, 15, or 45 μg of the recombinant SARS-CoV-2 S protein(s) per dose, optionally wherein each dose of the immunogenic composition is 0.25 mL in volume without adjuvant, or 0.5 mL in volume with adjuvant.

16. The immunogenic composition of claim 15, wherein each dose of the composition comprises a total of 5 μg of the recombinant SARS-CoV-2 S protein(s), optionally wherein the composition comprises two different recombinant SARS-CoV-2 S proteins in equal amounts, optionally wherein each dose of the immunogenic composition is 0.25 mL in volume without adjuvant, or 0.5 mL in volume with adjuvant.

17. The immunogenic composition of claim 15, wherein each dose of the composition comprises a total of 10 μg of the recombinant SARS-CoV-2 S protein(s), optionally wherein the composition comprises two different recombinant SARS-CoV-2 S proteins in equal amounts, optionally wherein each dose of the immunogenic composition is 0.25 mL in volume without adjuvant, or 0.5 mL in volume with adjuvant.

18. The immunogenic composition of claim 12, comprising a recombinant SARS-CoV-2 S protein comprising residues 19-1243 of SEQ ID NO: 10 and/or a recombinant SARS-CoV-2 S protein comprising residues 19-1240 of SEQ ID NO: 14.

19. A container containing the immunogenic composition of claim 12.

20. The container of claim 19, wherein the container is a vial or a syringe.

21. The container of claim 19, wherein the container contains a single dose or multiple doses of the immunogenic composition.

22. A kit for intramuscular vaccination, wherein the kit comprises two containers, wherein a first container contains a pharmaceutical composition comprising one, two, three, or more of the recombinant SARS-CoV-2 S proteins of claim 5, and a second container contains an adjuvant.

23. The kit of claim 22, wherein the first container comprises one or more antigen doses, wherein each antigen dose comprises a total of about 2 to 45 μg of the recombinant SARS-CoV-2 S protein(s) provided in 0.25 mL of a phosphate-buffered saline (PBS), optionally wherein the PBS comprises
(i) 7.5 mM phosphate and 150 mM NaCl, pH 7.2, and optionally 0.2% polysorbate 20, or
(ii) 0.097 mg sodium phosphate monobasic monohydrate, 0.26 mg sodium phosphate dibasic anhydrous, 2.2 mg sodium chloride, 550 μg polysorbate 20, and qs. ad 0.25 mL water.

24. The kit of claim 23, wherein each antigen dose comprises a total of 2.5, 5, 10, 15, or 45 μg of recombinant SARS-CoV-2 S protein(s), optionally wherein the antigen dose comprises
(i) a recombinant SARS-CoV-2 S protein comprising residues 19-1243 of SEQ ID NO:10,
(ii) a recombinant SARS-CoV-2 S protein comprising residues 19-1240 of SEQ ID NO:14, or
(iii) both (i) and (ii).

25. The kit of claim 22, wherein the second container comprises one or more doses of the adjuvant, wherein each dose of the adjuvant is 0.25 mL in volume and comprises:
12.5 mg squalene,
1.85 mg sorbitan monooleate,
2.38 mg polyoxyethylene cetostearyl ether, and
2.31 mg mannitol
in a PBS, optionally wherein the PBS comprises or is prepared by mixing
(i) 7.5 mM phosphate and 150 mM NaCl, pH 7.2, and optionally a polysorbate, optionally polysorbate 20; or
(ii) 0.097 mg sodium phosphate monobasic monohydrate, 0.26 mg dibasic sodium phosphate anhydrous, 2.2 mg sodium chloride, 50-600, optionally 55 or 550, μg polysorbate, optionally polysorbate 20, and qs. ad 0.25 mL water.

26. A method of making a vaccine kit, comprising:
providing the immunogenic composition of claim 12, and
packaging the composition into a sterile container.

27. A method of preventing or ameliorating COVID-19 in a subject in need thereof, comprising administering to the subject a prophylactically effective amount of the immunogenic composition of claim 12.

28. A method of preventing or ameliorating COVID-19 in a subject in need thereof, comprising administering to the subject a prophylactically effective amount of the immunogenic composition of claim 12, wherein prior to the administering step, the subject has been infected by SARS-CoV-2 or has been vaccinated with a first COVID-19 vaccine.

29. The method of claim 28, wherein prior to the administering step, the subject has been vaccinated with a genetic, subunit, or killed vaccine.

30. The method of claim 29, wherein prior to the administering step, the subject has been vaccinated with a genetic vaccine comprising an mRNA that encodes a recombinant SARS-CoV-2 S antigen.

31. The method of claim 28, wherein the administering step takes place 4 weeks, one month, three months, four months, five months, six months, seven months, eight months, nine months, ten months, eleven months, or one year, optionally four to ten months, further optionally eight months, post-infection or after the subject is vaccinated with the first COVID-19 vaccine, optionally wherein the immunogenic composition comprises 2.5 or 5 μg of each of the recombinant SARS-CoV-2 S protein(s), and further optionally wherein the immunogenic composition is monovalent or multivalent.

32. The method of claim 27, wherein the prophylactically effective amount is about 2 to 50 μg per dose, optionally 2.5, 5, 10, 15, or 45 μg per dose.

33. The method of claim 27, wherein the prophylactically effective amount is administered in a single dose or in two or more doses, optionally intramuscularly.

34. The method of claim 33, comprising administering to the subject two doses of the immunogenic composition with an interval of about two weeks to about three months, wherein each dose of the immunogenic composition comprises a total of 2.5, 5 or 10 μg of the recombinant SARS-CoV-2 S protein(s).

35. The method of claim 34, wherein the interval is about three weeks or about 21 days, or about four weeks or about 28 days, or about one month.

36. The method of claim 27, wherein the subject is a human subject, optionally wherein the human subject is a child, an adult, or an elderly adult.

37-38. (canceled)
